(19) United States
(12) Patent Application Publication
(10) Pub. No.: US 2021/0341756 A1
(43) Pub. Date: Nov. 4, 2021

(54) INTERFERENCE PATTERN ABLATION SYSTEMS AND METHODS

(71) Applicant: TRUIRIS LLC, Columbus, OH (US)

(72) Inventors: Jefferson ODHNER, Amherst, NH (US); Robin SEARS, Columbus, OH (US)

(21) Appl. No.: 17/243,403

(22) Filed: Apr. 28, 2021

Related U.S. Application Data
(60) Provisional application No. 63/017,590, filed on Apr. 29, 2020.

(51) Int. Cl. G02C 7/04; A61B 3/113

(52) U.S. Cl. CPC G02C 7/046; A61B 3/113

(57) ABSTRACT

Provided herein are embodiments of systems and methods for imparting a pattern or representation to a device using interference pattern ablation.

### CROSS-REFERENCE TO RELATED APPLICATIONS

This application claims the benefit of U.S. Provisional Application No. 63/017,590 filed Apr. 29, 2020, which application is incorporated herein by reference for all purposes in its entirety.

### BACKGROUND

Interference pattern ablation may find use in a number of applications, such as imparting patterns onto a device surface. Prior systems and methods of patterned ablations may be less than desirable in some respects.

### SUMMARY

Provided herein are embodiments of a system for imparting a pattern onto a surface, the system comprising: a laser for emitting a laser beam along an optical path to the surface; and an aperture substrate comprising one or more aperture patterns to be placed in the optical path; wherein emission of the laser beam is coordinated with a position of the aperture substrate such that the laser beam is modified by the one or more aperture patterns to impart at least a portion of the pattern onto the surface.

In some embodiments, the system further comprises an encoder configured to track the position of the aperture substrate. In some embodiments, the aperture substrate is an aperture wheel. In some embodiments, the aperture wheel is configured to rotate. In some embodiments, a motor rotates the aperture wheel. In some embodiments, the system further comprises a controller for coordinating the emission of the laser beam with the position of the aperture substrate.

In some embodiments, the surface is a surface of a wearable ocular device. In some embodiments, the wearable ocular device is a contact lens. In some embodiments, the surface is a front surface of the contact lens. In some embodiments, the surface is a back surface of the contact lens. In some embodiments, the contact lens is a dehydrated hydrogel contact lens.

In some embodiments, the one or more aperture patterns are configured to account for an expansion of the dehydrated hydrogel contact lens during hydration. In some embodiments, the wearable ocular device comprises at least one area devoid of patterning. In some embodiments, there is at least one area devoid of patterning corresponds to a location of a pupil of a wearer. In some embodiments, the at least one area devoid of patterning comprises a diameter of 1 millimeter to 5 millimeters.

In some embodiments, the system further comprises a focal lens comprised of one or more optical elements to focus the modified laser beam onto the surface. In some embodiments, the focal lens is placed into the optical path after the laser beam is modified by the one or more aperture patterns.

In some embodiments, the system further comprises a zoom lens comprised of one or more optical elements to magnify the pattern. In some embodiments, the zoom lens is placed into the optical path after the laser beam is modified by the one or more aperture patterns.

In some embodiments, the system further comprises a beam expander comprised of one or more optical elements. In some embodiments, the beam expander is placed into the optical path prior to the aperture wheel.

In some embodiments, the laser is a pulsed laser. In some embodiments, the laser is a continuous wave laser.

In some embodiments, the surface is provided on a moveable stage.

In some embodiments, the pattern is a representation. In some embodiments, the representation is an expression or designation. In some embodiments, the expression or designation is a geometric object. In some embodiments, the geometric object comprises a dot, a line, a triangle, a quadrilateral, a rectangle, a square, a pentagon, a hexagon, a heptagon, an octagon, a nonagon, a decagon, an undecagon, a dodecagon, a polygon with more than 12 sides, an ellipse, an oval, or a circle. In some embodiments, the expression or designation provides an indication as to whether the device is properly centered or oriented on a wearer of the device. In some embodiments, the expression or designation is a repository of information about the device. In some embodiments, the repository of information comprises a barcode, a QR code, or a QR code with a circular hole in the center. In some embodiments, the repository of information is used to track the device during manufacturing or during an ophthalmological study or clinical trial. In some embodiments, the expression or designation is a character or a term. In some embodiments, the expression or designation is an image. In some embodiments, the image comprises a symbol, a logo, a brand, a photograph, a work of art, or a cartoon. In some embodiments, the image is obtained through a scanning procedure. In some embodiments, the expression or designation is configured to alter an appearance of a wearer of the device for an artistic purpose. In some embodiments, the expression or designation is a color.

Provided herein are embodiments of a system for imparting a pattern onto a surface, the system comprising: a laser for emitting a laser beam along an optical path to the surface; a plurality of rotatable aperture wheels, each aperture wheel comprising one or more aperture patterns to be placed in the optical path; and an encoder system configured to track the position of the aperture wheel, wherein emission of the laser beam is synchronized with rotation of the plurality of aperture wheels such that the laser beam is modified by the one or more aperture patterns to impart at least a portion of the pattern onto the surface.

In some embodiments, each rotatable aperture wheel comprises a window such that light passing through the window is not further modified.

Provided herein are embodiments of a method for imparting a pattern on to a surface, comprising: tracking a rotation of an aperture wheel having one or more aperture patterns; selecting an aperture pattern of the one or more aperture patterns to modify the laser beam; and emitting the laser beam along an optical path from a laser to the surface when the first aperture pattern is aligned with the optical path.

In some embodiments, the one or more aperture patterns are configured to modify a laser beam into a light pattern. In some embodiments, the method further comprises a step of applying an optically absorptive material to the surface prior to the step of emitting the laser beam. In some embodiments, the method further comprises a step of removing the optically absorptive material.

In some embodiments, the method further comprises repeating steps of the method to impart a plurality of patterns onto the surface.

In some embodiments, the pattern comprises a diffraction grating. In some embodiments, the surface is a surface of a wearable ocular device. In some embodiments, the wearable ocular device is a contact lens. In some embodiments, the surface is a front surface of the contact lens. In some embodiments, the surface is a back surface of the contact lens. In some embodiments, the contact lens is a dehydrated hydrogel contact lens. In some embodiments, the method further comprises a step of adding an aqueous solution to the dehydrated hydrogel contact lens. In some embodiments, the aqueous solution comprises water.

In some embodiments, the method further comprises a step of focusing the pattern onto the surface. In some embodiments, the method further comprises a step of magnifying the pattern onto the surface.

Provided herein are embodiments of a composition of a contact lens comprising: a silicone hydrogel substrate; and an optically absorptive layer applied to the surface of the silicone hydrogel substrate.

In some embodiments, the silicone hydrogel substrate comprises a siloxane macromer. In some embodiments, the silicone hydrogel substrate comprises a hydrophilic monomer. In some embodiments, the hydrophilic monomer comprises hydroxyethyl methacrylate, poly-hydroxyethyl methacrylate, dimethylaminoethyl methacrylate, or a combination thereof.

In some embodiments, the silicon hydrogel substrate comprises approximately 30% to 80% water by weight when hydrated. In some embodiments, the silicon hydrogel substrate comprises approximately 30% to 40% water by weight when hydrated. In some embodiments, the silicon hydrogel substrate comprises approximately 50% to 60% water by weight when hydrated. In some embodiments, the silicon hydrogel substrate comprises approximately 60% to 80% water by weight when hydrated.

In some embodiments, the optically absorptive layer comprises a thickness of approximately 1 nanometer to 1000 micrometers. In some embodiments, the optically absorptive layer comprises a thickness of approximately 100 nanometer to 500 nanometers. In some embodiments, the optically absorptive layer comprises a thickness of approximately 500 nanometer to 1 micrometer. In some embodiments, the optically absorptive layer comprises a thickness of approximately 1 micrometer to 100 micrometers. In some embodiments, the optically absorptive layer comprises a thickness of approximately 100 micrometers to 500 micrometers.

In some embodiments, the silicon hydrogel substrate comprises methyl bis(trimethylsiloxy)silyl propyl glycerol methacrylate. In some embodiments, the silicon hydrogel substrate comprises galyfilcon, senofilcon, or a combination thereof. In some embodiments, the silicon hydrogel substrate comprises galyfilcon, senofilcon, or a combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A illustrates a front view of a wearable ocular device comprising a diffraction grating, in accordance with some embodiments;

FIG. 1B illustrates a side view of a wearable ocular device comprising a diffraction grating, in accordance with some embodiments;

FIG. 1C illustrates a first color chart of colors that may be imparted to a device surface using the systems and methods described herein;

FIG. 1D illustrates a second color chart of colors that may be imparted device surface using the systems and methods described herein;

FIG. 2 depicts a system for imparting a pattern onto a device surface using the methods described herein;

FIG. 3 depicts a system for imparting a pattern onto a device surface using the methods described herein;

FIG. 4 illustrates a flowchart for a method of imparting a representation to a surface of a device, in accordance with some embodiments;

FIG. 5 illustrates a flowchart for a method of imparting a representation to a surface of a device, in accordance with some embodiments;

FIG. 6 illustrates a flowchart for a method of imparting a representation to a surface of a device, in accordance with some embodiments; and

FIG. 7 illustrates a computer system that is programmed or otherwise configured to operate any of the systems or methods described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

In some embodiments, the systems and methods disclosed herein are provided to impart a patterned ablation onto a surface of a device. The device may be an optical device, security device, mold, imprinting master, cosmetic device, or other device suitable for patterned ablation. In some embodiments, an optical device includes a diffraction grating or other light modification device used in an optics setting. In some embodiments, a security device includes a holographic security tag, tamper-evident security tag, or other security device which may be used for authentication. In some embodiments, a cosmetic device includes a wearable cosmetic device, ocular cosmetic device, or other cosmetic device for representing a particular aesthetic or design.

I. Wearable Ocular Device

As used herein, the term “wearable ocular device” may comprise any ocular device that may be worn by a user. For instance, a wearable ocular device may comprise a contact lens. A wearable ocular device may comprise bifocals. A wearable ocular device may comprise an ocular prosthesis.

Reference will now be made to the figures, wherein like numerals may refer to like characters throughout. It will be appreciated that the figures are not necessarily drawn to scale.

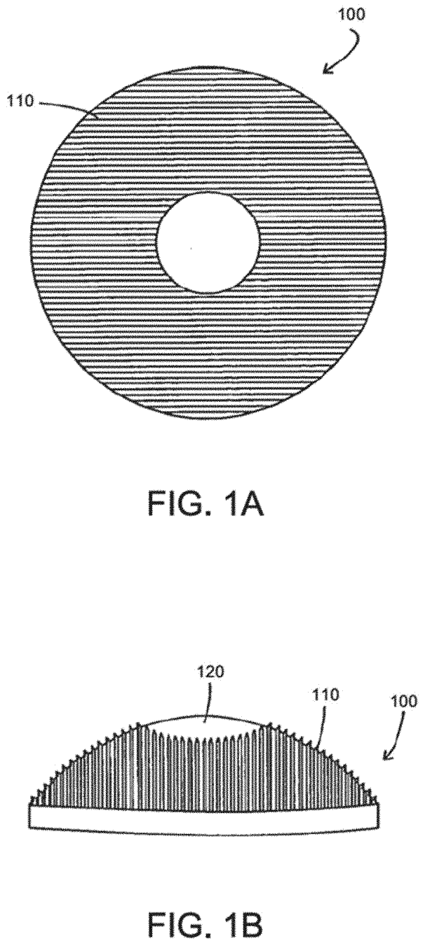

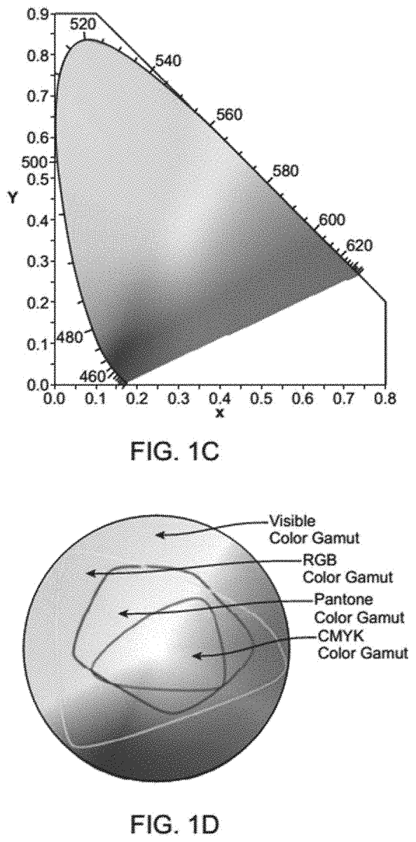

FIG. 1A depicts a front view of an embodiment of a colored wearable ocular device 100 comprising a diffraction grating. As depicted in FIG. 1A, the device may comprise a contact lens. The contact lens may comprise a soft contact lens. The contact lens may comprise a disposable soft contact lens. The contact lens may comprise a soft daily contact lens. The contact lens may comprise a soft extended wear contact lens. The contact lens may comprise a hard contact lens. The contact lens may comprise a rigid gas permeable contact lens. The contact lens may comprise a hybrid contact lens. The contact lens may comprise a spherical lens. The contact lens may comprise a toric lens. The contact lens may comprise a monovision lens. The contact lens may comprise a bifocal lens. The contact lens may comprise a multifocal lens. Though depicted in FIG. 1A as a contact lens, the device 100 may comprise any wearable ocular device described herein. The device may comprise bifocals. The device may comprise an ocular prosthesis.

The ocular prosthesis may comprise an artificial eye. The ocular prosthesis may replace an absent natural eye. For instance, the ocular prosthesis may replace an absent natural eye following an enucleation, evisceration, orbital exenteration, or other removal of a natural eye. The ocular prosthesis may be shaped to fit under a user's eyelid. The ocular prosthesis may be shaped to fit over an orbital implant. The ocular prosthesis may comprise a convex shell shape. The ocular prosthesis may comprise a thin hard shell (e.g. a scleral shell) to be worn over a damaged eye. The ocular prosthesis may comprise a spherical shape. The ocular prosthesis may comprise a non-spherical shape. The ocular prosthesis may comprise a conical orbital implant (COI) or a multi-purpose conical orbital implant (MCOI). The ocular prosthesis may comprise a pyramid implant. The ocular prosthesis may comprise a flat surface. The ocular prosthesis may comprise preformed channels for rectus muscles of an eye. The ocular prosthesis may comprise a recessed slot for a superior rectus of an eye. The ocular prosthesis may comprise a protrusion to fill a superior fornix of an eye. The ocular prosthesis may comprise a conical shape that closely the anatomic shape of an ocular orbit. The ocular prosthesis may comprise a relatively wide anterior portion. The ocular prosthesis may comprise a relatively narrow posterior portion.

The ocular prosthesis may comprise a non-integrated implant. The ocular prosthesis may comprise a non-integrated spherical intraconal implant. The ocular prosthesis may comprise an integrated implant. The ocular prosthesis may comprise a quasi-integrated implant. The ocular prosthesis may comprise a coupling device. The ocular prosthesis may comprise a surface configured to improve implant motility of the ocular prosthesis. The ocular prosthesis may comprise an insert to accommodate a round-headed peg or screw. The round-headed peg or screw may transfer implant motility to the ocular prosthesis. The ocular prosthesis may be configured to allow for fibrovascular ingrowth following implantation of the ocular prosthesis.

The ocular prosthesis may comprise a glass eye. The ocular prosthesis may comprise a cryolite glass. The ocular prosthesis may comprise a sodium hexafluoroaluminate (Na₃AlF₆) glass. The ocular prosthesis may comprise a plastic. The ocular prosthesis may comprise a thermoplastic. The ocular prosthesis may comprise one or more materials selected from the group consisting of: polymethylmethacrylate (PMMA), hydroxyapatite (HA), polyethylene (PE), high density polyethylene, porous polyethylene (PP), high density porous polyethylene (Medpor), polyethylene terephthalate (PET), vicryl, silicone, and a bioceramic (such as aluminum oxide, Al₂O₃).

The device 100 may comprise a diffraction grating 110 applied to a surface of the device. The surface of the device may be a front surface of a contact lens. The surface of the device may be a back surface of a contact lens. The diffraction grating may be configured to impart a representation to the device. The representation may be an expression or a designation.

The expression or designation may be a geometric object. For instance, the diffraction grating may cause a viewer of the device to perceive one or more dots, lines, shapes (such as one or more triangles, quadrilaterals, rectangles, squares, pentagons, hexagons, heptagons, octagons, nonagons, decagons, undecagons, dodecagons, polygons with more than 12 sides, ellipses, ovals, circles, or any other geometric shape). Such markings may represent an indication of one or more optically relevant parameters of the device, such as whether the device is properly centered or oriented on a wearer of the device. In some cases, the markings may represent an indication of whether a contact lens is properly centered or oriented on an eye of a wearer of the contact lens. For instance, the marking may comprise a bump or lenticular that indicates an orientation of the contact lens.

The expression or designation may be a repository of information. For instance, the diffraction grating may cause a viewer of the device to perceiver a barcode, a QR code, or a QR code with a circular hole in its center. In some embodiments, the circular hole is provided as to not obscure the vision of a wearer. In some embodiments, the circular hole corresponds to a pupil of the wearer. In some embodiments, the circular hole is approximately 1 to 5 mm in diameter. The repository of information may be useful for quality control or other tracking purposes. For instance, the repository of information may enable tracking of the device during manufacturing or during an ophthalmological study or clinical trial.

The expression or designation may be a character or term. The character or term may be a character or term selected from any language, such as Mandarin, Spanish, English, Hindi, Arabic, Portuguese, Bengali, Russian, Japanese, Punjabi, German, Javanese, Wu, Malay, Telugu, Vietnamese, Korean, French, Marathi, Tamil, Urdu, Turkish, Italian, Yue, Cantonese, Thai, Gujarati, Jin, Min, Persian, Polish, Pashto, Kannada, Xiang, Malayalam, Sundanese, Hausa, Odia, Burmese, Hakka, Ukrainian, Bhojpuri, Tagalog, Yoruba, Maithili, Uzbek, Sindhi, Amharic, Fula, Romanian, Oromo, Igbo, Azerbaijani, Awadhi, Gan, Cebuano, Dutch, Kurdish, Serbo-Croatian, Malagasy, Saraiki, Nepali, Sinhalese, Chittagonian, Zhuang, Khmer, Turkmen, Assamese, Madurese, Somali, Marwari, Magahi, Haryanvi, Hungarian, Chhattisgarhi, Greek, Chewa, Deccan, Akan, Kazakh, Sylheti, Zulu, Czech, Kinyarwanda, Dhundhari, Haitian, Creole, Ilocano, Quechua, Kirundi, Swedish, Hmong, Shona, Uyghur, Hiligaynon, Ilonggo, Mossi, Xhosa, Belarusian, Balochi, Konkani, or any other language.

The expression or designation may be an image, such as one or more logos, brands, photographs, works of art, cartoons, or other images. The image may be obtained through an image scanning procedure.

The expression or designation may be configured to alter an appearance of a wearer of the device for artistic purposes, such as for use in movies or other live action performances. In some cases, the expression or designation may be configured to alter an appearance of an eye of a wearer of a contact lens for artistic purposes. For instance, the expression or designation may alter the appearance of the wearer's eye such that the wearer appears to have the eyes of an animal, monster, or other non-human.

The expression or designation may be a color. In such a case, the diffraction grating may be configured to impart a desired color to the device. The diffraction grating may have the effect of taking light that strikes the diffraction grating and diffracting that light into multiple colors. The colors may be dispersed widely in angular space for a tightly spaced diffraction grating. The colors may be dispersed narrowly in angular space for a less tightly spaced diffraction grating. An observer who views the device may perceive the color of the device as a color of the rainbow which depends on the observer's viewing angle and the angle from which illumination light strikes the diffraction grating.

The pattern or expression may comprise one or more areas which do not comprise any patterning, diffraction grating elements, or other features. In some embodiments, the areas without patterning or features may provide a transparent window as to not obscure the vision of a wearer. In some embodiments, the area is void of patterning comprise an optical element to enhance or correct the vision of a wearer. In some embodiments, the optical element to enhance or correct the vision of a wearer is implemented based on a prescribed vision correction procedure.

In some embodiments, the one or more areas devoid of patterning or diffraction grating structures defines a clear boundary area about a pupil of a wearer. Said areas may correlate to a pupil area, such that vision of a wearer is not obscured by the diffraction grating or pattern imparted onto the device. In some embodiments, the area devoid of patterning is 1 millimeter (mm) to 5 mm in diameter. In some embodiments, the area without patterning is positioned to correspond to a location of a pupil of the wearer. In some embodiments, the area without patterning is circular. In some embodiments, the area without patterning is substantially centered on the ocular device.

In some embodiments, an area free from patterning comprises a diameter of about 1 mm to about 10 mm. In some embodiments, an area free from patterning comprises a diameter of about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 6 mm to about 10 mm, about 7 mm to about 8 mm, about 7 mm to about 9 mm, about 7 mm to about 10 mm, about 8 mm to about 9 mm, about 8 mm to about 10 mm, or about 9 mm to about 10 mm. In some embodiments, an area free from patterning comprises a diameter of about 1 mm, about 2 mm, about 3 mm, about 4 mm, about 5 mm, about 6 mm, about 7 mm, about 8 mm, about 9 mm, or about 10 mm. In some embodiments, an area free from patterning comprises a diameter of at least about 1 mm, about 2 mm, about 3 mm, about 4 mm, about 5 mm, about 6 mm, about 7 mm, about 8 mm, or about 9 mm. In some embodiments, an area free from patterning comprises a diameter of at most about 2 mm, about 3 mm, about 4 mm, about 5 mm, about 6 mm, about 7 mm, about 8 mm, about 9 mm, or about 10 mm.

The expression or designation may be an artificial pupil. The artificial pupil may comprise one or more moth eye structures.

The diffraction grating may be designed using a variety of optical parameters, such as how tightly the diffraction grating is spaced. By careful selection of the optical parameters, the diffraction grating may be designed such that an observer perceives a rainbow of colors or the observer perceives a single color over a wide angle. The diffraction grating may be a simple grating, a compound grating, a blazed grating, or a pattern of grating dots.

FIG. 1B shows a side view of a colored wearable ocular device 100 comprising a diffraction grating. As shown in FIG. 1B, the device may be designed such that a wearer of the device does not perceive a change in the wearer's vision due to the presence of the diffraction grating 110. The diffraction grating may be annular in shape so as to leave a transparent region 120 of the device over a wearer's iris, allowing light to pass through a lens of a wearer's eye and strike the wearer's retina.

The device 100 may comprise a plurality of diffraction gratings applied to the surface of the device. The device may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, or more diffraction gratings applied to the surface of the device. The device may comprise at most 100, at most 90, at most 80, at most 70, at most 60, at most 50, at most 40, at most 30, at most 20, at most 19, at most 18, at most 17, at most 16, at most 15, at most 14, at most 13, at most 12 at most 11, at most 10, at most 9, at most 8, at most 7, at most 6, at most 5, at most 4, at most 3, at most 2, or fewer diffraction gratings applied to the surface of the device. The device may comprise a number of diffraction gratings that is within a range defined by any two of the preceding values applied to the surface of the device. Any two or more of the diffraction gratings may be arranged at any angle to one another. For instance, any two or more of the diffraction gratings may be arranged at an angle of at least 1 degree, at least 2 degrees, at least 3 degrees, at least 4 degrees, at least 5 degrees, at least 6 degrees, at least 7 degrees, at least 8 degrees, at least 9 degrees, at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 81 degrees, at least 82 degrees, at least 83 degrees, at least 84 degrees, at least 85 degrees, at least 86 degrees, at least 87 degrees, at least 88 degrees, at least 89 degrees, or more, to one another. Any two or more of the diffraction gratings may be arranged at an angle of at most 90 degrees, at most 89 degrees, at most 88 degrees, at most 87 degrees, at most 86 degrees, at most 85 degrees, at most 84 degrees, at most 83 degrees, at most 82 degrees, at most 81 degrees, at most 80 degrees, at most 75 degrees, at most 70 degrees, at most 65 degrees, at most 60 degrees, at most 55 degrees, at most 50 degrees, at most 45 degrees, at most 40 degrees, at most 35 degrees, at most 30 degrees, at most 25 degrees, at most 20 degrees, at most 15 degrees, at most 10 degrees, at most 9 degrees, at most 8 degrees, at most 7 degrees, at most 6 degrees, at most 5 degrees, at most 4 degrees, at most 3 degrees, at most 2 degrees, at most 1 degrees, or less, to one another. Any two of more of the diffraction gratings may be arranged at an angle that is within a range defined by any two of the preceding values.

For instance, the device 100 may comprise first, second, and third diffraction gratings applied to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from an International Commission on Illumination (CIE) color chart such as that described herein with respect to FIG. 1C or a condensed CIE color chart such as that described herein with respect to FIG. 1D. The desired color may be detected through the use of an optical spectrometer or a digital camera. The desired color may correspond to the color of an iris or pupil of one or more eyes of a wearer of the device.

In another example, the device 100 may comprise first, second, third, fourth, fifth, and sixth diffraction gratings applied to the surface of the device. The first and fourth diffraction gratings may form a cross grating pair. For instance, the first and fourth diffraction gratings may be substantially perpendicular to one another. The first and fourth diffraction grating may have optical parameters selected such that they impart the same or a similar color to the device, such as a red hue. Similarly, the second and fifth diffraction gratings may form a cross grating pair. For instance, the second and fifth diffraction gratings may be substantially perpendicular to one another. The second and fifth diffraction grating may have optical parameters selected such that they impart the same or a similar color to the device, such as a green hue. The third and sixth diffraction gratings may form a cross grating pair. For instance, the third and sixth diffraction gratings may be substantially perpendicular to one another. The third and sixth diffraction grating may have optical parameters selected such that they impart the same or a similar color to the device, such as a blue hue. The use of cross gratings may increase the efficiency of the optical effects produced by the gratings.

The diffraction grating 110 may be produced by any of the methods described herein, such as any of methods 400, 500, and 600 described herein. For instance, the diffraction grating may be imprinted on the surface of the device. The diffraction grating may comprise a plurality of regions that have been ablated from the surface of the device. The diffraction grating may comprise a lithographically patterned phase change material, such as a lithographically patterned photopolymer.

FIG. 1C shows a first color chart of colors that may be imparted to a wearable ocular device using the systems and methods described herein. The color chart may comprise a CIE color chart. The CIE color chart may be used to select a color to be imparted to a wearable ocular device described herein using any of the methods described herein.

FIG. 1D shows a second color chart of colors that may be imparted to a wearable ocular device using the systems and methods described herein. The color chart may comprise a condensed CIE color chart. The condensed CIE color chart may be used to select a color to be imparted to a wearable ocular device described herein using any of the methods described herein.

Wearable ocular devices of the present disclosure may have therapeutic applications. For instance, the device 100 may provide corneal protection for wearers that suffer conditions such as entropion, trichiasis, tarsal scarring, recurrent corneal erosion, or post-surgical ptosis. The device 100 may provide corneal pain relief for wearers that suffer conditions such as bullous keratopathy, epithelial erosion, epithelial abrasion, filamentary keratitis, or post-keratoplasty. The device 100 may be used as a bandage during a healing process for conditions such as chronic epithelial defect, corneal ulcer, neurotrophic keratitis, neuroparalytic keratitis, chemical burn, or post-surgical epithelial defect. The device 100 may be used as a bandage during a healing process after an ocular surgery such as small incision lenticule extraction (SMILE), laser-assisted in situ keratomileusis (LASIK), laser epithelial keratomileusis (LASEK), photorefractive keratectomy (PRK), penetrating keratoplasty (PK), phototherapeutic keratectomy (PTK), automated lamellar keratoplasty (ALK), refractive lens exchange (RLE), presbyopic lens exchange (PRELEX), lamellar graft, corneal flap, or other corneal surgical conditions. The device 100 may be used to provide optical correction during a healing procedure if such optical correction is necessary or desired.

The device 100 may be used to mask or camouflage a condition such as aniridia, pupil irregularity, permanent eye damage, or amblyopia in order to improve the appearance of a wearer of the device or to improve quality of life for a wearer of the device. The device 100 may be used to mitigate or eliminate double vision or to mitigate or eliminate the need for an occluder lens. In such an application, the device 100 may comprise a solid black pupillary component on an inner portion of the device (which may have a diameter of 1-4 mm larger that a maximum pupil size of an eye of a wearer of the device) to block out light and a clear outer edge on an outer portion of the device. The diameter of the solid black pupillary component may be selected based on measurements of the maximum size of the pupil obtained in dim light conditions. The device 100 may be used to mitigate or eliminate photophobia. In such an application, the device 100 may comprise a prosthetic iris lens in an inner portion of the device (thereby mitigating or eliminating light sensitivity) and a clear outer edge on an outer portion of the device. The prosthetic iris lens may have a diameter that is large enough to ensure coverage of the disfigured iris of a wearer of the device.

The device 100 may be used to enhance contrast or vision. For instance, the device 100 may be used to create a sunglass effect whereby brightness of light received by an eye of a wearer of the device is reduced. The device 100 may be used to increase or maximize contrast by applying a color tint (such as a gray, green, or amber tint) to the device. Such contrast-enhanced devices may be particularly useful to athletes in enhancing their athletic performance. The device 100 may be used to correct color vision deficiencies, such as by providing a red tint to the device.

II. Systems for Patterned Ablation

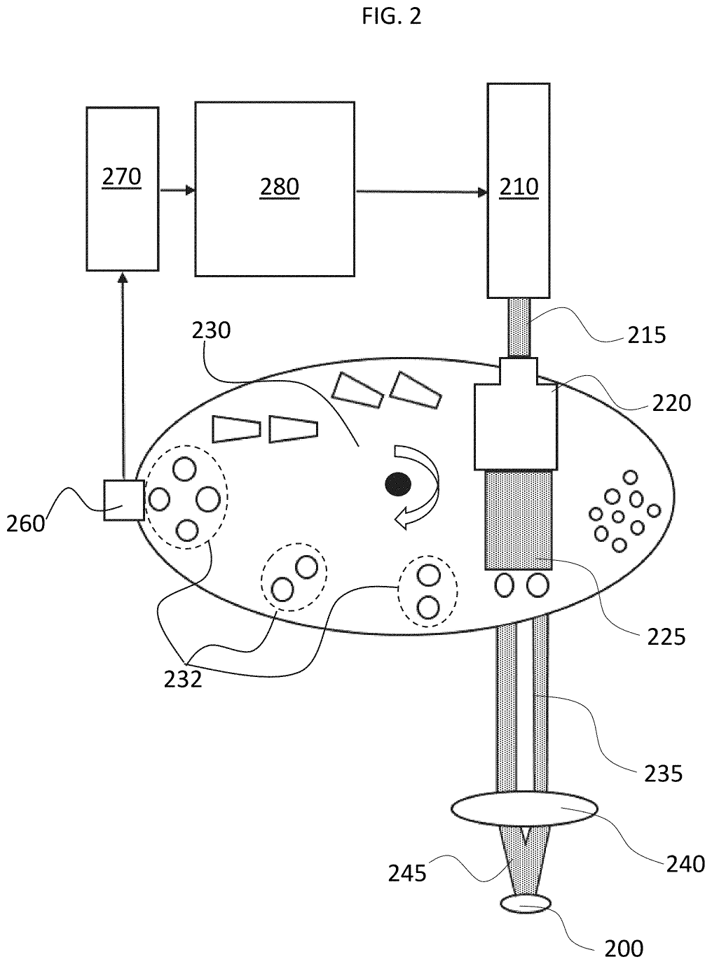

FIG. 2 depicts a system for imparting a pattern onto a surface 200 of a device according to some embodiments. In some embodiments, the system comprises a laser 210 directed toward the surface 200, such that laser beam 215 travels along an optical path from the laser 210 to the surface 200.

In some embodiments, an aperture substrate is provided along the optical path of the laser beam 215. The aperture substrate may comprise multiple aperture patterns 232. Each of the aperture patterns may include one or more apertures to create a specific interference pattern as the laser beam passes through the apertures and constructive and destructive interference occurs on the other side of the aperture substrate. The substrate may comprise aperture patterns to create complex gratings in a single shot such as 4 apertures in a square configuration to create a cross grating, or in a rectangular configuration creating a cross grating with two different spatial frequencies. In some embodiments, the aperture substrate may comprise two or more similar aperture patterns with different spacing between the apertures of the pattern such that spatial frequencies of the patterns applied to the surface 200 are varied. In some embodiments, a closer spacing between apertures of an aperture pattern 232 creates a larger spatial frequency, while apertures that are further apart create higher spatial frequencies when of a pattern imparted onto a surface 200. In some embodiments, the aperture substrate comprises a substantially planar surface with one or more aperture patterns provided through the surface. The aperture substrate may be moved or repositioned with respect to the laser beam to select an aperture pattern to create an interference pattern. The interference pattern may be imparted onto a surface 200 of a device. In some embodiments the interference pattern is ablated onto the surface of the device.

In some embodiments, the aperture substrate is provided as a rotatable wheel 230. In some embodiments, the aperture wheel 230 may comprise two or more similar aperture patterns with different spacing between the apertures of the pattern such that spatial frequencies of the patterns applied to the surface 200 are varied. In some embodiments, a closer spacing between apertures of an aperture pattern 232 creates a larger spatial frequency, while apertures that are further apart create higher spatial frequencies when of a pattern imparted onto a surface 200.

In some embodiments, the system further comprises a laser beam expander 220. The beam expander 220 may be provided to expand laser beam 215 creating an expanded beam 225. In some embodiments, the expanded beam 225 comprises a beam diameter large enough to pass light through all apertures of an aperture pattern 232.

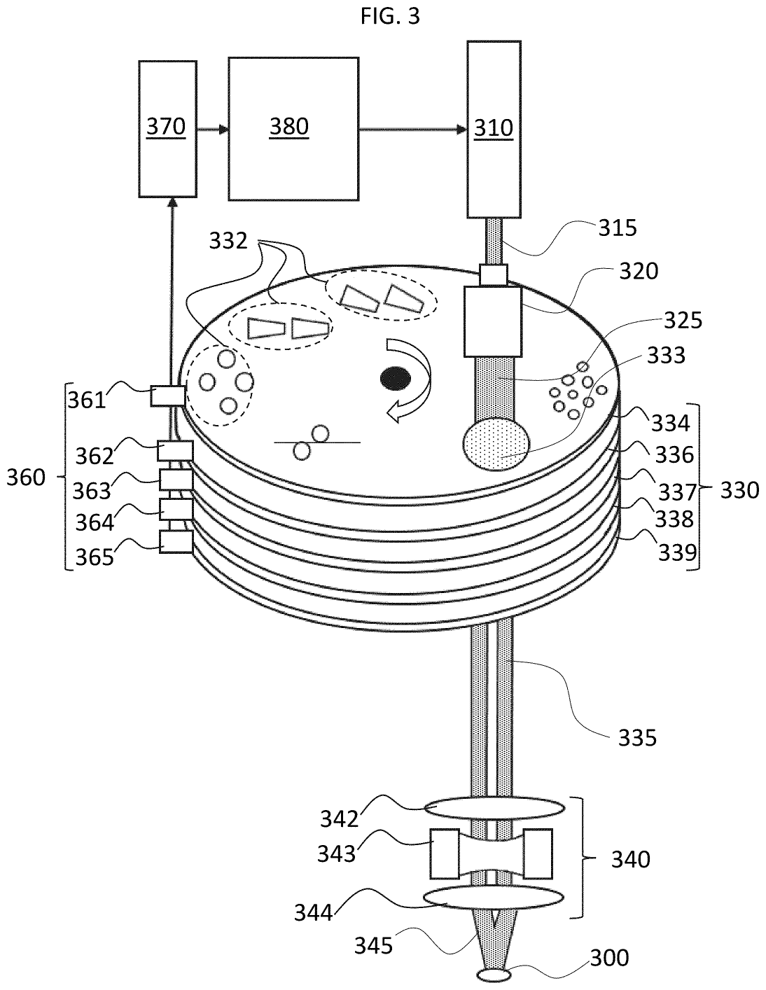

In some embodiments, the system is further provided with a converging or focal optical system 240. In some embodiments, the optical system may be a zoom optical configuration (e.g. 340 as depicted in FIG. 3). In some embodiments, the converging lens 240 combines light from multiple apertures of an aperture pattern 232 onto the surface 200 where the interference occurs, creating an ablation pattern. In some embodiments, the focal lens 240 focuses the interference pattern onto the surface 200 to create an ablation pattern. In some embodiments. In some embodiments, the converging lens 240 combines light from multiple apertures of an aperture pattern 232 onto the surface 200 where the interference occurs, creating a holographic grating. In some embodiments, the focal lens 240 focuses the interference pattern onto the surface 200 to create a holographic grating.

In some embodiments, the system further comprises an encoder 260 to track the position of the aperture wheel 230 as it rotates. In some embodiments, the encoder comprises an optical encoder. The optical encoder may track the position of the aperture wheel 230 by sensing one or more markings near the circumference of the aperture wheel 230 via an optical sensor. In some embodiments, the encoder comprises an electric or magnetic encoder, wherein the encoder tracks the position of the aperture wheel 230 by sensing one or more instances of a ferromagnetic or conductive material deposited near the circumference of the aperture wheel 230.

In some embodiments, the system further includes a computing system 270. The computing system may be configured to receive signals from the encoder 260 and track the position of the aperture wheel. In some embodiments, the computing device 270 is further configured to synchronize a beam emission from the laser 210, such that a laser beam emitted by the laser is passed through a desired aperture pattern 232.

In some embodiments, the system further comprises a controller or laser pulse trigger 280 to control the emission of the laser beam 215 from laser 210. In some embodiments, the controller receives a signal from computing device 270 which initiates emission of the laser beam 215. In some embodiments, when the encoder indicates the desired aperture pattern 232 is in the optical path of the laser beam 215, the laser 210 fires to create a grating. In some embodiments, the controller 280 is further configured to control rotation of the aperture wheel 230. In some embodiments, the controller 280 is in communication with a motor which spins to aperture wheel 230. The controller 280 may adjust the angular velocity of the aperture wheel to better synchronize pulsing of the laser 210 alignment of the desired aperture pattern 232.

In some embodiments, the device is provided on a movable stage, such the surface 200 may be moved relative to the converged beam 245 to enable patterned ablation of the entire surface 200. In such embodiments, the stage may move the device such that multiple ablation patterns are created across the surface 200. In some embodiments, the stage is moveable in an XY plane (orthogonal to the laser beam). In some embodiments, the stage is moveable in a Z-direction (parallel to the laser beam). In some embodiments, the stage is tiltable. In some embodiments, the stage is able to tilt about an X-axis at a theta (0) angle. In some embodiments, the stage is able to tilt about a Y-axis at a Phi (1) angle.

In an example, a laser 210 may operate at a frequency of 100 Hz while the aperture wheel rotates rapidly. With the laser operating at 100 Hz, the system may impart 100 ablation patterns per second onto the surface 200 of the device. When the encoder 260 indicates the desired aperture pattern 232 is aligned with the laser 210, the laser may be trigger by controller 280 to emit beam 215 such that a pattern is created on the surface 200. The device 200 may be provided on a movable stage. The movable stage may move the device continuously. The laser pulse width may be about 4 nanoseconds; therefore, the stage may appear stationary relative to the short laser pulse width, therefore the stage can be moved continuously as the patterns are ablated onto a surface 200 of the device.

In some embodiments, multiple ablation patterns are created on the surface 200 of the device to form a visual representation, as described herein. The device may be a wearable ocular device, as described herein. The device may be an optical device, security device, mold, imprinting master, cosmetic device, or other device suitable for patterned ablation. In some embodiments, an optical device includes a diffraction grating or other light modification device used in an optics setting. In some embodiments, a security device includes a holographic security tag, tamper-evident security tag, or other security device which may be used for authentication. In some embodiments, a cosmetic device includes a wearable cosmetic device, ocular cosmetic device, or other cosmetic device for representing a particular aesthetic or design.

FIG. 3 depicts a system for imparting a pattern onto a surface 300 of a device according to some embodiments. In some embodiments, the system comprises a laser 310 directed toward the surface 300, such that laser beam 315 travels along an optical path from the laser 310 to the surface 300.

some embodiments, the system comprises a plurality of aperture substrates, each having one or more aperture patterns. The aperture substrates may be positioned to place a selected aperture pattern in the optical path of the laser beam to create an interference pattern. The interference pattern may be imparted onto a surface 300 of a device. In some embodiments the interference pattern is ablated onto the surface of the device.

In some embodiments, the aperture substrates comprise a plurality of aperture wheels 330 provided along the optical path of the laser beam 315. Each aperture wheel (334, 336, 337, 338, 339) of the plurality of aperture wheels 330 may comprise multiple aperture patterns 332. Each of the aperture patterns 332 may include one or more apertures to create a specific interference pattern as the laser beam passes through the apertures and produces constructive and destructive interference during convergence.

FIG. 3 depicts a plurality of aperture wheels 330 comprised of five aperture wheels (334, 336, 337, 338, 339), however a plurality of aperture wheels used in the system may comprise any number of aperture wheels to provide various aperture patterns as necessary. In some embodiments, the system comprises 1 aperture wheel to 10 aperture wheels. In some embodiments, the system comprises 1 aperture wheel to 2 aperture wheels, 1 aperture wheel to 3 aperture wheels, 1 aperture wheel to 4 aperture wheels, 1 aperture wheel to 5 aperture wheels, 1 aperture wheel to 6 aperture wheels, 1 aperture wheel to 7 aperture wheels, 1 aperture wheel to 8 aperture wheels, 1 aperture wheel to 9 aperture wheels, 1 aperture wheel to 10 aperture wheels, 2 aperture wheels to 3 aperture wheels, 2 aperture wheels to 4 aperture wheels, 2 aperture wheels to 5 aperture wheels, 2 aperture wheels to 6 aperture wheels, 2 aperture wheels to 7 aperture wheels, 2 aperture wheels to 8 aperture wheels, 2 aperture wheels to 9 aperture wheels, 2 aperture wheels to 10 aperture wheels, 3 aperture wheels to 4 aperture wheels, 3 aperture wheels to 5 aperture wheels, 3 aperture wheels to 6 aperture wheels, 3 aperture wheels to 7 aperture wheels, 3 aperture wheels to 8 aperture wheels, 3 aperture wheels to 9 aperture wheels, 3 aperture wheels to 10 aperture wheels, 4 aperture wheels to 5 aperture wheels, 4 aperture wheels to 6 aperture wheels, 4 aperture wheels to 7 aperture wheels, 4 aperture wheels to 8 aperture wheels, 4 aperture wheels to 9 aperture wheels, 4 aperture wheels to 10 aperture wheels, 5 aperture wheels to 6 aperture wheels, 5 aperture wheels to 7 aperture wheels, 5 aperture wheels to 8 aperture wheels, 5 aperture wheels to 9 aperture wheels, 5 aperture wheels to 10 aperture wheels, 6 aperture wheels to 7 aperture wheels, 6 aperture wheels to 8 aperture wheels, 6 aperture wheels to 9 aperture wheels, 6 aperture wheels to 10 aperture wheels, 7 aperture wheels to 8 aperture wheels, 7 aperture wheels to 9 aperture wheels, 7 aperture wheels to 10 aperture wheels, 8 aperture wheels to 9 aperture wheels, 8 aperture wheels to 10 aperture wheels, or 9 aperture wheels to 10 aperture wheels. In some embodiments, the system comprises 1 aperture wheel, 2 aperture wheels, 3 aperture wheels, 4 aperture wheels, 5 aperture wheels, 6 aperture wheels, 7 aperture wheels, 8 aperture wheels, 9 aperture wheels, or 10 aperture wheels. In some embodiments, the system comprises at least 1 aperture wheel, 2 aperture wheels, 3 aperture wheels, 4 aperture wheels, 5 aperture wheels, 6 aperture wheels, 7 aperture wheels, 8 aperture wheels, or 9 aperture wheels. In some embodiments, the system comprises at most 2 aperture wheels, 3 aperture wheels, 4 aperture wheels, 5 aperture wheels, 6 aperture wheels, 7 aperture wheels, 8 aperture wheels, 9 aperture wheels, or 10 aperture wheels. The aperture substrates may also take the form of other substantially planar surfaces having one or more aperture patterns in place of the aperture wheels as depicted and disclosed herein.

The wheel may comprise aperture patterns to create complex gratings in a single shot such as 4 apertures in a square configuration to create a cross grating, or in a rectangular configuration creating a cross grating with two different spatial frequencies. In some embodiments, the plurality of aperture wheels 330 may comprise two or more similar aperture patterns with different spacing between the apertures of the pattern such that spatial frequencies of the patterns applied to the surface 300 are varied. In some embodiments, a closer spacing between apertures of an aperture pattern 332 creates a larger spatial frequency, while apertures that are further apart create higher spatial frequencies when of a pattern imparted onto a surface 300.

In some embodiments, wherein the system comprises a plurality of aperture wheels 330, each aperture wheel (334, 336, 337, 338, 339) of the plurality of aperture wheels 330 comprises a clear aperture or window. In some embodiments, the window is a through hole in the aperture wheel. In some embodiments, the diameter of each window is larger than the diameter surrounding any aperture pattern provided on any one of the aperture wheels. According to some embodiments, FIG. 3 depicts window 333 provided by aperture wheel 334. Similarly, aperture wheels 336, 337, 338, and 339 may also include a window (not shown). In some embodiments, the window 333 does not modify the laser beam or light pattern as it passes through the aperture wheel it is provided on. In some embodiments, each aperture wheel (334, 336, 337, 338, 339) of the plurality of aperture wheels 330 comprises a clear aperture.

In some embodiments, the system further comprises a laser beam expander 320. The beam expander 320 may be provided to expand laser beam 315 creating an expanded beam 325. In some embodiments, the expanded beam 325 comprises a beam diameter large enough to pass light through all apertures of an aperture pattern 332.

In some embodiments, the system is further provided with an optical system 340 having a focusing lens with zoom capability. In some embodiments, the optical system is simply a focal lens (e.g. 240 as depicted in FIG. 2). In some embodiments, the focusing lens 340 combines light from multiple apertures of an aperture pattern 332 onto the surface 300 where the interference occurs, creating an ablation pattern. In some embodiments, the focal lens 340 focuses the interference pattern onto the surface 300 to create an ablation pattern. In some embodiments. In some embodiments, the converging lens 340 combines light from multiple apertures of an aperture pattern 332 onto the surface 300 where the interference occurs, creating a holographic grating. In some embodiments, the focal lens 340 focuses the interference pattern onto the surface 300 to create a holographic grating. In some embodiments, the zoom capability of the focal lens 340 allows for the size or magnification of the to be varied. In some embodiments, the zoom of the focal lens 340 is varied between ablations of multiple patterns.

In some embodiments, a zoom capability may be provided by three lenses. In some embodiments, zoom capability is provided by an afocal system comprising of two positive (converging) lenses 342,344 of equal focal length with a negative (diverging) lens 343 between them. In some embodiments, the absolute focal length of the diverging lens 343 is less than half that of the positive lenses 342,344. In some embodiments, lens 344 is fixed, but lenses 342 and 343 can be moved axially in a particular non-linear relationship. In some embodiments, While the diverging lens 343 moves toward the fixed lens 344, the moving converging lens 342 moves forward and then backward in a parabolic arc.

In some embodiments, the system further comprises an encoder 360 system to track the position of the plurality of aperture wheels 330 as they rotate. In some embodiments, each aperture wheel (334, 336, 337, 338, and 339) is provided with an encoder (361, 362, 363, 364, and 365, respectively) In some embodiments, encoders comprise an optical encoders. The optical encoders may track the position of the associated aperture wheel by sensing one or more markings near the circumference of the aperture wheel via an optical sensor. In some embodiments, the encoders comprise electric or magnetic encoders, wherein the encoders track the position of the associated aperture wheel by sensing one or more instances of a ferromagnetic or conductive material deposited near the circumference of the aperture wheel.

In some embodiments, the system further includes a computing system 370. The computing system may be configured to receive signals from the encoder system 360 and track the position of the aperture wheels. In some embodiments, the computing device 370 is further configured to synchronize a beam emission from the laser 310, such that a laser beam emitted by the laser is passed through the desired aperture patterns 332 or windows 333 of the aperture wheels.

In some embodiments, the system further comprises a controller or laser pulse trigger 380 to control the emission of the laser beam 315 from laser 310. In some embodiments, the controller receives a signal from computing device 370 which initiates emission of the laser beam 315. In some embodiments, when the encoder system indicates the desired aperture patterns 332 or windows 333 are in the optical path of the laser beam 315, the laser 310 fires to create a desired light pattern to be emitted onto the surface 300 of a device. In some embodiments, the controller 380 is further configured to control rotation of each aperture wheel (334, 336, 337, 338, and 339) the plurality of aperture wheels 330. In some embodiments, the controller 380 is in communication with a motor system which spins the aperture wheels 330. The controller 380 may adjust the angular velocity of the aperture wheel to better synchronize pulsing of the laser 310 alignment of the desired aperture pattern 332. In some embodiments, each aperture wheel (334, 336, 337, 338, and 339) is individually controlled. In some embodiments, each aperture wheel (334, 336, 337, 338, and 339) is provided with a motor to control the angular velocity of the aperture wheel.

In some embodiments, the device is provided on a movable stage, such the surface 300 may be moved relative to the converged beam 345 to enable patterned ablation of the entire surface 300. In such embodiments, the stage may move the device such that multiple ablation patterns are created across the surface 300. In some embodiments, the stage is moveable in an XY plane (orthogonal to the laser beam). In some embodiments, the stage is moveable in a Z-direction (parallel to the laser beam). In some embodiments, the stage is tiltable. In some embodiments, the stage is able to tilt about an X-axis at a theta (0) angle. In some embodiments, the stage is able to tilt about a Y-axis at a Phi (1) angle.

In an example, a laser 310 may operate at a frequency of 100 Hz while the aperture wheels rotate rapidly. With the laser operating at 100 Hz, the system may impart 100 ablation patterns per second onto the surface 300 of the device. When the encoder system 360 indicates the desired aperture patterns 332 or windows 333 are aligned with the laser 310, the laser may be trigger by controller 380 to emit beam 315 such that a pattern is created on the surface 300. The device 300 may be provided on a movable stage. The movable stage may move the device continuously. The laser pulse width may be about 4 nanoseconds; therefore, the stage may appear stationary relative to the short laser pulse width, therefore the stage can be moved continuously as the patterns are ablated onto a surface 300 of the device.

In some embodiments, only one aperture pattern of one of the aperture wheels is utilized to modify the laser beam to produce an ablation pattern. In some embodiments, the aperture pattern selected to modify the laser beam belonging to a particular aperture wheel is synchronized with the windows (e.g. 333 of aperture wheel 334) of the other aperture wheels such that the laser beam is only modified by the selected aperture pattern. In some embodiments, aperture patterns provided on multiple wheels are aligned to produce complex patterns.

In some embodiments, multiple ablation patterns are created on the surface 300 of the device to form a visual representation, as described herein. The device may be a wearable ocular device, as described herein. The device may be an optical device, security device, mold, imprinting master, cosmetic device, or other device suitable for patterned ablation. In some embodiments, an optical device includes a diffraction grating or other light modification device used in an optics setting. In some embodiments, a security device includes a holographic security tag, tamper-evident security tag, or other security device which may be used for authentication. In some embodiments, a cosmetic device includes a wearable cosmetic device, ocular cosmetic device, or other cosmetic device for representing a particular aesthetic or design.

In some embodiments, rotation of each aperture wheel is individually variable. In some embodiments, an aperture wheel rotates at about 100 rotations per minute (RPM) to about 10,000 RPM. In some embodiments, an aperture wheel rotates at about 100 RPM to about 1,000 RPM, about 100 RPM to about 3,000 RPM, about 100 RPM to about 5,000 RPM, about 100 RPM to about 6,000 RPM, about 100 RPM to about 10,000 RPM, about 1,000 RPM to about 3,000 RPM, about 1,000 RPM to about 5,000 RPM, about 1,000 RPM to about 6,000 RPM, about 1,000 RPM to about 10,000 RPM, about 3,000 RPM to about 5,000 RPM, about 3,000 RPM to about 6,000 RPM, about 3,000 RPM to about 10,000 RPM, about 5,000 RPM to about 6,000 RPM, about 5,000 RPM to about 10,000 RPM, or about 6,000 RPM to about 10,000 RPM. In some embodiments, an aperture wheel rotates at about 100 RPM, about 1,000 RPM, about 3,000 RPM, about 5,000 RPM, about 6,000 RPM, or about 10,000 RPM. In some embodiments, an aperture wheel rotates at least at about 100 RPM, about 1,000 RPM, about 3,000 RPM, about 5,000 RPM, or about 6,000 RPM, including increments therein. In some embodiments, an aperture wheel rotates at most at about 1,000 RPM, about 3,000 RPM, about 5,000 RPM, about 6,000 RPM, or about 10,000 RPM, including increments therein.

III. Methods of Patterned Ablation

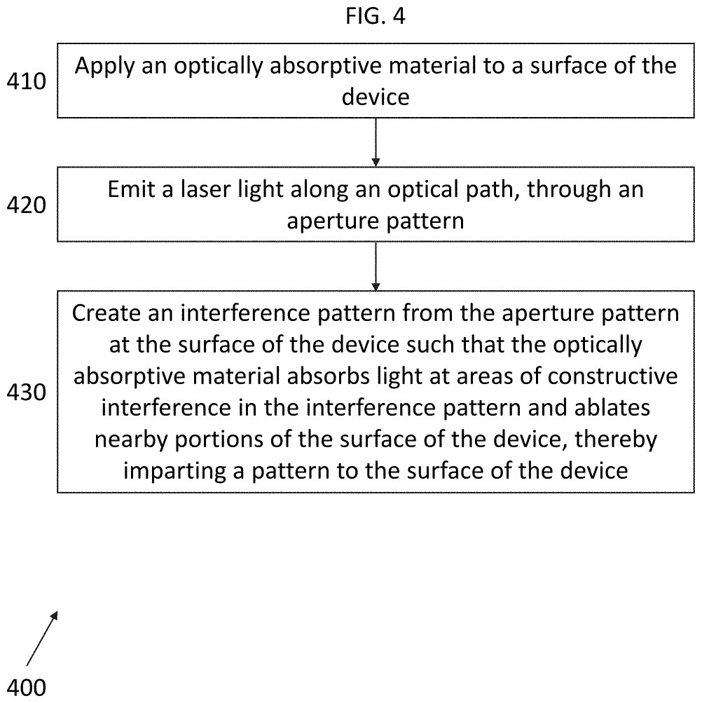

FIG. 4 shows a flowchart for a method 400 of imparting a representation to a surface of a device using aperture interference ablation to produce a diffraction grating or ablation pattern on a surface of the device. In a first operation 410, the method 400 may comprise applying an optically absorptive material to a surface of the device. The device may be any device described herein.

In a second operation 420, the method 400 may comprise emitting a laser light along an optical path and through an aperture pattern. The laser light may be any laser light as described herein. The aperture pattern may be provided on an aperture wheel as described herein.

In a third operation 430, the method 400 may comprise creating an interference pattern from the laser light passing through the aperture pattern and projecting the interference pattern incident on the surface of the device such that the optically absorptive material absorbs light at areas of constructive interference in the interference pattern and ablates nearby portions of the surface of the device, thereby imparting a pattern to the surface of the device. The pattern may comprise a diffraction grating. The method 400 may be used to impart any representation described herein (such as any representation described herein with respect to FIG. 1A, 1B, 1C, or 1D) to the device. The representation may be an expression or designation.

The method 400 may further comprise repeating any 1, 2, or 3 of operations 410, 420, and 430 to impart a plurality of diffraction gratings to the surface of the device. The method 400 may further comprise repeating any 1, 2, or 3 of operations 410, 420, and 430 at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 400 may further comprise repeating any 1, 2, or 3 of operations 410, 420, and 430 at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 400 may further comprise repeating any 1, 2, or 3 of operations 410, 420, and 430 a number of times that is within a range defined by any two of the preceding values to impart a number of diffraction gratings that is within a range defined by any two of the preceding values to the surface of the device. In some embodiments, the device is provided on a movable stage which repositions the surface of the device relative to the optical path between repetitions of ablating patterns on the surface of the device.

For instance, the method 200 may further comprise repeating any 1, 2, or 3 of operations 410, 420, and 430 a total of three times to impart first, second, and third ablation patterns to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 400 may further comprise removing the optically absorptive material from the surface of the device.

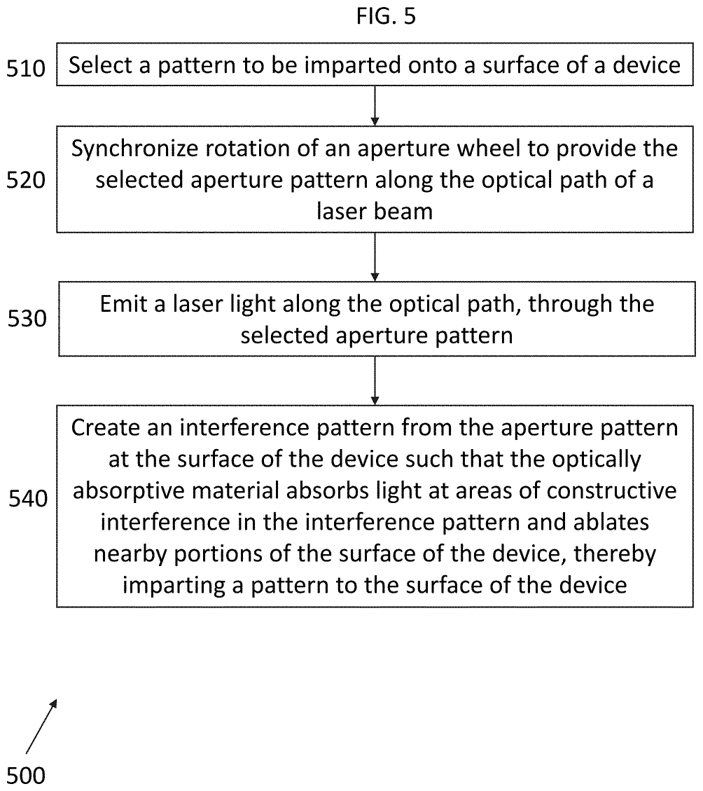

FIG. 5 shows a flowchart for a method 500 of imparting a representation to a surface of a device using aperture interference ablation to produce a patterned ablation on a surface of the device. In a first operation 510, the method 500 may comprise selecting a pattern to be imparted to the surface of the device. The device may be a wearable ocular device. The device may be a contact lens. The contact lens may be any contact lens described herein. The device may be bifocals. The device may be an ocular prosthesis. The device may be any device disclosed herein.

In a second operation 520, the method 500 may comprise synchronizing or adjusting rotation of an aperture wheel to provide an aperture pattern along the optical path of the laser beam, where the aperture pattern is configured to produce a selected ablation pattern on a surface of the device. The surface of the device may be a front surface of a contact lens. The surface of the device may be a back surface of a contact lens.

In a third operation 530, the method 500 may comprise emitting a laser light along the optical path, such that it passes through the selected aperture pattern to produce the selected ablation pattern.

In a fourth operation 540, the method 500 may comprise creating an interference pattern incident on the surface of the device such that the optically absorptive material absorbs light at areas of constructive interference in the interference pattern and ablates nearby portions of the surface of the device, thereby imparting a pattern to the surface of the device. The laser light may be similar to any laser light described herein.

The surface of the device may be configured such that a normal to the surface of the device makes an angle with the laser light. The surface of the device may be configured such that a normal to the surface of the device makes an angle of at least 1 degree, at least 2 degrees, at least 3 degrees, at least 4 degrees, at least 5 degrees, at least 6 degrees, at least 7 degrees, at least 8 degrees, at least 9 degrees, at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 81 degrees, at least 82 degrees, at least 83 degrees, at least 84 degrees, at least 85 degrees, at least 86 degrees, at least 87 degrees, at least 88 degrees, at least 89 degrees, or more, with the laser light. The surface of the device may be configured such that a normal to the surface of the device makes an angle of at most 90 degrees, at most 89 degrees, at most 88 degrees, at most 87 degrees, at most 86 degrees, at most 85 degrees, at most 84 degrees, at most 83 degrees, at most 82 degrees, at most 81 degrees, at most 80 degrees, at most 75 degrees, at most 70 degrees, at most 65 degrees, at most 60 degrees, at most 55 degrees, at most 50 degrees, at most 45 degrees, at most 40 degrees, at most 35 degrees, at most 30 degrees, at most 25 degrees, at most 20 degrees, at most 15 degrees, at most 10 degrees, at most 9 degrees, at most 8 degrees, at most 7 degrees, at most 6 degrees, at most 5 degrees, at most 4 degrees, at most 3 degrees, at most 2 degrees, at most 1 degrees, or less, with the laser light. The surface of the device may be configured such that a normal to the surface of the device makes an angle that is within a range of any two of the preceding values, with the laser light.

The optical path may comprise a spatial filter. The spatial filter may comprise a lens.

The method 500 may be used to impart any representation described herein (such as any representation described herein with respect to FIG. 1A, 1B, 1C, or 1D) to the device. The representation may be an expression or designation.

The method 500 may further comprise repeating any 1, 2, 3, or 4 of operations 510, 520, 530, and 540 to impart a plurality of diffraction gratings to the surface of the device. The method 500 may further comprise repeating any 1, 2, 3, or 4 of operations 510, 520, 530, and 540 at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 500 may further comprise repeating any 1, 2, 3, or 4 of operations 510, 520, 530, and 540 at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 500 may further comprise repeating any 1, 2, 3, or 4 of operations 510, 520, 530, and 540 a number of times that is within a range defined by any two of the preceding values to impart a number of diffraction gratings that is within a range defined by any two of the preceding values to the surface of the device.

For instance, the method 500 may further comprise repeating any 1, 2, 3, or 4 of operations 510, 520, 530, and 540 a total of three times to impart first, second, and third diffraction gratings to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 500 may further comprise removing the optically absorptive material from the surface of the device.

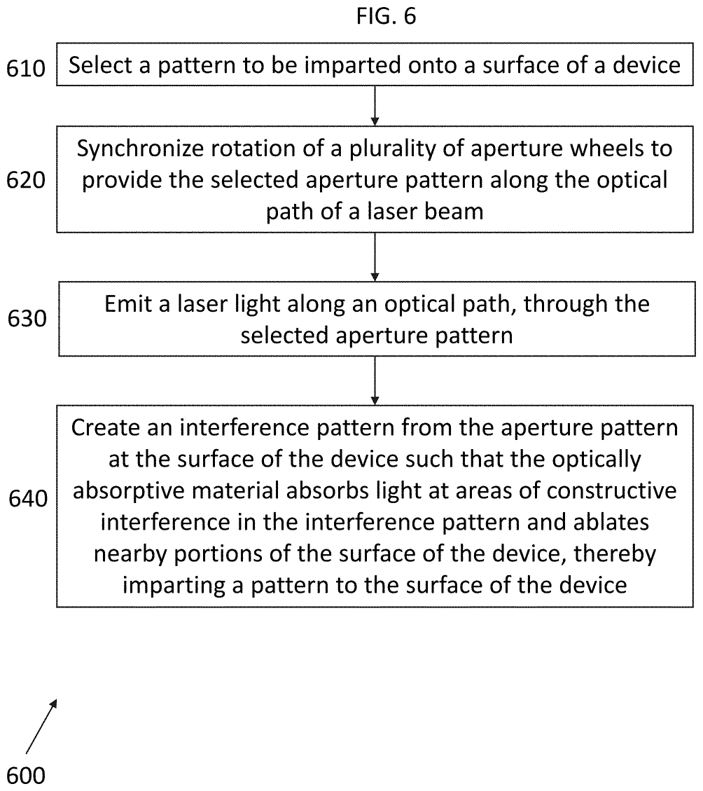

FIG. 6 shows a flowchart for a method 600 of imparting a representation to a wearable ocular device using reflection holography ablation to produce a patterned ablation on a surface of the device. In a first operation 610, the method 600 may comprise selecting a pattern to be imparted to the surface of the device. The device may be a contact lens. The contact lens may be any contact lens described herein. The device may be bifocals. The device may be an ocular prosthesis.

In a second operation 620, the method 600 may comprise synchronizing or adjusting rotation of one or more aperture wheels to provide a select aperture pattern along the optical path of the laser beam, where the aperture pattern is configured to produce a selected ablation pattern on a surface of the device. The second operation 620, may further comprise synchronizing or adjusting the other aperture wheels, not containing the select aperture pattern, such that the laser beam is not modified prior to passing through the select aperture pattern, and the select ablation pattern is not modified after passing through the select aperture pattern. The surface of the device may be a front surface of a contact lens. The surface of the device may be a back surface of a contact lens.

In a third operation 630, the method 600 may comprise emitting a laser light along the optical path, such that it passes through the selected aperture pattern to produce the selected ablation pattern.

In a fourth operation 640, the method 600 may comprise creating an interference pattern incident on the surface of the device such that the optically absorptive material absorbs light at areas of constructive interference in the interference pattern and ablates nearby portions of the surface of the device, thereby imparting a pattern to the surface of the device. The laser light may be similar to any laser light described herein.

The surface of the device may be configured such that a normal to the surface of the device makes an angle with the laser light. The surface of the device may be configured such that a normal to the surface of the device makes an angle of at least 1 degree, at least 2 degrees, at least 3 degrees, at least 4 degrees, at least 5 degrees, at least 6 degrees, at least 7 degrees, at least 8 degrees, at least 9 degrees, at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 81 degrees, at least 82 degrees, at least 83 degrees, at least 84 degrees, at least 85 degrees, at least 86 degrees, at least 87 degrees, at least 88 degrees, at least 89 degrees, or more, with the laser light. The surface of the device may be configured such that a normal to the surface of the device makes an angle of at most 90 degrees, at most 89 degrees, at most 88 degrees, at most 87 degrees, at most 86 degrees, at most 85 degrees, at most 84 degrees, at most 83 degrees, at most 82 degrees, at most 81 degrees, at most 80 degrees, at most 75 degrees, at most 70 degrees, at most 65 degrees, at most 60 degrees, at most 55 degrees, at most 50 degrees, at most 45 degrees, at most 40 degrees, at most 35 degrees, at most 30 degrees, at most 25 degrees, at most 20 degrees, at most 15 degrees, at most 10 degrees, at most 9 degrees, at most 8 degrees, at most 7 degrees, at most 6 degrees, at most 5 degrees, at most 4 degrees, at most 3 degrees, at most 2 degrees, at most 1 degrees, or less, with the laser light. The surface of the device may be configured such that a normal to the surface of the device makes an angle that is within a range of any two of the preceding values, with the laser light.

The optical path may comprise a spatial filter. The spatial filter may comprise a lens.

The method 600 may be used to impart any representation described herein (such as any representation described herein with respect to FIG. 1A, 1B, 1C, or 1D) to the device. The representation may be an expression or designation.

The method 600 may further comprise repeating any 1, 2, 3, or 4 of operations 610, 620, 630, and 640 to impart a plurality of diffraction gratings to the surface of the device. The method 600 may further comprise repeating any 1, 2, 3, or 4 of operations 610, 620, 630, and 640 at least 1 time, at least 2 times, at least 3 times, at least 4 times, at least 5 times, at least 6 times, at least 7 times, at least 8 times, at least 9 times, at least 10 times, or more, to impart at least 1, at least 2, at least 3, at least 4, at least 5, at least 6, at least 7, at least 8, at least 9, at least 10, or more diffraction gratings to the surface of the device. The method 600 may further comprise repeating any 1, 2, 3, or 4 of operations 610, 620, 630, and 640 at most 10 times, at most 9 times, at most 8 times, at most 7 times, at most 6 times, at most 5 times, at most 4 times, at most 3 times, at most 2 times, or less, to impart at most 10, at most 9, at most 8, at most 7, at most 6, at most 5, at most 4, at most 3, at most 2, or fewer diffraction gratings to the surface of the device. The method 600 may further comprise repeating any 1, 2, 3, or 4 of operations 610, 620, 630, and 640 a number of times that is within a range defined by any two of the preceding values to impart a number of diffraction gratings that is within a range defined by any two of the preceding values to the surface of the device.

For instance, the method 600 may further comprise repeating any 1, 2, 3, or 4 of operations 610, 620, 630, and 640 a total of three times to impart first, second, and third diffraction gratings to the surface of the device. The first diffraction grating may impart a red hue to the device. The second diffraction grating may impart a green hue to the device. The third diffraction grating may impart a blue hue to the device. The red, green, and blue hues may be chosen to impart a desired color to the device. The desired color may be chosen from a color chart, such as any color chart described herein. For instance, the desired color may be chosen from a CIE color chart such as that described herein with respect to FIG. 1C or a condensed CIE color chart such as that described herein with respect to FIG. 1D.

The method 600 may further comprise removing the optically absorptive material from the surface of the device.

IV. Ocular Devices

The device may be any device described herein. The device may be a contact lens. The surface of the device may be a front surface of the contact lens. The surface of the device may be a back surface of the contact lens. The device may be bifocals. The device may be an ocular prosthesis.

The ocular device may comprise a silicone hydrogel substrate. In some embodiments, the substrate is capable of being dehydrated. The silicone hydrogel substrate may comprise a siloxane macromer and/or a hydrophilic monomer. In some embodiments, the hydrophilic monomer comprises hydroxyethyl methacrylate, poly-hydroxyethyl methacrylate, dimethylaminoethyl methacrylate, or a combination thereof. The silicon hydrogel substrate may further comprise methyl bis(trimethylsiloxy)silyl propyl glycerol methacrylate (the Tanaka molecule) as a polar group to serve as an internal wetting agent. The silicon hydrogel substrate may comprise galyfilcon, senofilcon, or combinations thereof. The silicon hydrogel substrate comprises galyfilcon, senofilcon, or combinations thereof.

In some embodiments, the aperture patterns and therefore the patterns to be imparted onto a dehydrated ocular device are configured to account for the expansion which will be induced as the device is hydrated. In some embodiments, the radius of curvature of the ocular device may undergo slight changes as the device is hydrated. In some embodiments, the radius of curvature decreases as the device is hydrated.

In some embodiments, hydration results in both linear and radial expansion of the ocular device. In some embodiments, linear expansion comprises expansion of thickness of the ocular device (in a Z-direction). In some embodiments, radial expansion comprises expansion the diameter of the ocular device. In some embodiments, radial expansion comprises expansion of the length and width of the ocular device (across an XY plane). In some embodiments, linear expansion comprises expansion of the height of the grating features. In some embodiments, radial expansion comprises expansion of the length and width of the grating features.

In some embodiments, linear expansion of the dehydrated device may be estimated by the following first order approximation equation:

% of Linear Expansion=−0.9+0.5θ_{X}(% H₂O)

Elongation, a ratio of one linear hydrate dimension to the same dimension in the dry state, may be proportional to the % of linear expansion (% LE). The formula may be used to account for changes in the diameter, radius of curvature, and thickness of hydrogel ocular devices as a function of their water content. The optically absorptive material may absorb light and heat, resulting in the removal of material from the surface of the device by ablation or sublimation. The optically absorptive material may comprise an ink. The optically absorptive material may comprise a dye. The optically absorptive material may be a thin film. The optically absorptive material may be a thin film. The optically absorptive material may have a thickness of at least 1 nanometer (nm), at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, at least 500 nm, at least 600 nm, at least 700 nm, at least 800 nm, at least 900 nm, at least 1 micrometer (μm), at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, at least 900 μm, or at least 1,000 μm, or more. The optically absorptive material may have a thickness of at most 1,000 μm, at most 900 μm, at most 800 μm, at most 700 μm, at most 600 μm, at most 500 μm, at most 400 μm, at most 300 μm, at most 200 μm, at most 100 μm, at most 90 μm, at most 80 μm, at most 70 μm, at most 60 μm, at most 50 μm, at most 40 μm, at most 30 μm, at most 20 μm, at most 10 μm, at most 9 μm, at most 8 μm, at most 7 μm, at most 6 μm, at most 5 μm, at most 4 μm, at most 3 μm, at most 2 μm, at most 1 μm, at most 900 nm, at most 800 nm, at most 700 nm, at most 600 nm, at most 500 nm, at most 400 nm, at most 300 nm, at most 200 nm, at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, or less. The optically absorptive material may have a thickness that is within a range defined by any two of the preceding values.

V. Laser Systems

The laser or beam light may be emitted by a laser. The laser light may be emitted by a continuous wave laser. The laser light may be emitted by a pulsed laser. The laser light may be emitted by a gas laser, such as a helium-neon (HeNe) laser, an argon (Ar) laser, a krypton (Kr) laser, a xenon (Xe) ion laser, a nitrogen (N₂) laser, a carbon dioxide (CO₂) laser, a carbon monoxide (CO) laser, a transversely excited atmospheric (TEA) laser, or an excimer laser. For instance, the laser light may be emitted by an argon dimer (Ar₂) excimer laser, a krypton dimer (Kr₂) excimer laser, a fluorine dimer (F₂) excimer laser, a xenon dimer (Xe₂) excimer laser, an argon fluoride (ArF) excimer laser, a krypton chloride (KrCl) excimer laser, a krypton fluoride (KrF) excimer laser, a xenon bromide (XeBr) excimer laser, a xenon chloride (XeCl) excimer laser, or a xenon fluoride (XeF) excimer laser. The laser light may be emitted by a dye laser.

The laser light may be emitted by a metal-vapor laser, such as a helium-cadmium (HeCd) metal-vapor laser, a helium-mercury (HeHg) metal-vapor laser, a helium-selenium (HeSe) metal-vapor laser, a helium-silver (HeAg) metal-vapor laser, a strontium (Sr) metal-vapor laser, a neon-copper (NeCu) metal-vapor laser, a copper (Cu) metal-vapor laser, a gold (Au) metal-vapor laser, a manganese (Mn) metal-vapor, or a manganese chloride (MnCl₂) metal-vapor laser.

The laser light may be emitted by a solid-state laser, such as a ruby laser, a metal-doped crystal laser, or a metal-doped fiber laser. For instance, the laser light may be emitted by a neodymium-doped yttrium aluminum garnet (Nd:YAG) laser, a neodymium/chromium doped yttrium aluminum garnet (Nd/Cr:YAG) laser, an erbium-doped yttrium aluminum garnet (Er:YAG) laser, a neodymium-doped yttrium lithium fluoride (Nd:YLF) laser, a neodymium-doped yttrium orthovanadate (ND:YVO₄) laser, a neodymium-doped yttrium calcium oxoborate (Nd:YCOB) laser, a neodymium glass (Nd:glass) laser, a titanium sapphire (Ti:sapphire) laser, a thulium-doped ytrrium aluminum garnet (Tm:YAG) laser, a ytterbium-doped ytrrium aluminum garnet (Yb:YAG) laser, a ytterbium-doped glass (Yt:glass) laser, a holmium ytrrium aluminum garnet (Ho:YAG) laser, a chromium-doped zinc selenide (Cr:ZnSe) laser, a cerium-doped lithium strontium aluminum fluoride (Ce:LiSAF) laser, a cerium-doped lithium calcium aluminum fluoride (Ce:LiCAF) laser, a erbium-doped glass (Er:glass), an erbium-ytterbium-codoped glass (Er/Yt:glass) laser, a uranium-doped calcium fluoride (U:CaF₂) laser, or a samarium-doped calcium fluoride (Sm:CaF₂) laser.

The laser light may be emitted by a semiconductor laser or diode laser, such as a gallium nitride (GaN) laser, an indium gallium nitride (InGaN) laser, an aluminum gallium indium phosphide (AlGaInP) laser, an aluminum gallium arsenide (AlGaAs) laser, an indium gallium arsenic phosphide (InGaAsP) laser, a vertical cavity surface emitting laser (VCSEL), or a quantum cascade laser.

The laser light may be continuous wave laser light. The laser light may be pulsed laser light. The laser light may have a pulse length of at least 1 femtoseconds (fs), at least 2 fs, at least 3 fs, at least 4 fs, at least 5 fs, at least 6 fs, at least 7 fs, at least 8 fs, at least 9 fs, at least 10 fs, at least 20 fs, at least 30 fs, at least 40 fs, at least 50 fs, at least 60 fs, at least 70 fs, at least 80 fs, at least 90 fs, at least 100 fs, at least 200 fs, at least 300 fs, at least 400 fs, at least 500 fs, at least 600 fs, at least 700 fs, at least 800 fs, at least 900 fs, at least 1 picosecond (ps), at least 2 ps, at least 3 ps, at least 4 ps, at least 5 ps, at least 6 ps, at least 7 ps, at least 8 ps, at least 9 ps, at least 10 ps, at least 20 ps, at least 30 ps, at least 40 ps, at least 50 ps, at least 60 ps, at least 70 ps, at least 80 ps, at least 90 ps, at least 100 ps, at least 200 ps, at least 300 ps, at least 400 ps, at least 500 ps, at least 600 ps, at least 700 ps, at least 800 ps, at least 900 ps, at least 1 nanosecond (ns), at least 2 ns, at least 3 ns, at least 4 ns, at least 5 ns, at least 6 ns, at least 7 ns, at least 8 ns, at least 9 ns, at least 10 ns, at least 20 ns, at least 30 ns, at least 40 ns, at least 50 ns, at least 60 ns, at least 70 ns, at least 80 ns, at least 90 ns, at least 100 ns, at least 200 ns, at least 300 ns, at least 400 ns, at least 500 ns, at least 600 ns, at least 700 ns, at least 800 ns, at least 900 ns, at least 1,000 ns, or more. The laser light may have a pulse length of at most 1,000 ns, at most 900 ns, at most 800 ns, at most 700 ns, at most 600 ns, at most 500 ns, at most 400 ns, at most 300 ns, at most 200 ns, at most 100 ns, at most 90 ns, at most 80 ns, at most 70 ns, at most 60 ns, at most 50 ns, at most 40 ns, at most 30 ns, at most 20 ns, at most 10 ns, at most 9 ns, at most 8 ns, at most 7 ns, at most 6 ns, at most 5 ns, at most 4 ns, at most 3 ns, at most 2 ns, at most 1 ns, at most 900 ps, at most 800 ps, at most 700 ps, at most 600 ps, at most 500 ps, at most 400 ps, at most 300 ps, at most 200 ps, at most 100 ps, at most 90 ps, at most 80 ps, at most 70 ps, at most 60 ps, at most 50 ps, at most 40 ps, at most 30 ps, at most 20 ps, at most 10 ps, at most 9 ps, at most 8 ps, at most 7 ps, at most 6 ps, at most 5 ps, at most 4 ps, at most 3 ps, at most 2 ps, at most 1 ps, at most 900 fs, at most 800 fs, at most 700 fs, at most 600 fs, at most 500 fs, at most 400 fs, at most 300 fs, at most 200 fs, at most 100 fs, at most 90 fs, at most 80 fs, at most 70 fs, at most 60 fs, at most 50 fs, at most 40 fs, at most 30 fs, at most 20 fs, at most 10 fs, at most 9 fs, at most 8 fs, at most 7 fs, at most 6 fs, at most 5 fs, at most 4 fs, at most 3 fs, at most 2 fs, at most 1 fs, or less. The laser light may have a pulse length that is within a range defined by any two of the preceding values. For instance, the laser light may have a pulse length between 1 ns and 50 ns.

The laser light may have a repetition rate of at least 1 hertz (Hz), at least 2 Hz, at least 3 Hz, at least 4 Hz, at least 5 Hz, at least 6 Hz, at least 7 Hz, at least 8 Hz, at least 9 Hz, at least 10 Hz, at least 20 Hz, at least 30 Hz, at least 40 Hz, at least 50 Hz, at least 60 Hz, at least 70 Hz, at least 80 Hz, at least 90 Hz, at least 100 Hz, at least 200 Hz, at least 300 Hz, at least 400 Hz, at least 500 Hz, at least 600 Hz, at least 700 Hz, at least 800 Hz, at least 900 Hz, at least 1 kilohertz (kHz), at least 2 kHz, at least 3 kHz, at least 4 kHz, at least 5 kHz, at least 6 kHz, at least 7 kHz, at least 8 kHz, at least 9 kHz, at least 10 kHz, at least 20 kHz, at least 30 kHz, at least 40 kHz, at least 50 kHz, at least 60 kHz, at least 70 kHz, at least 80 kHz, at least 90 kHz, at least 100 kHz, at least 200 kHz, at least 300 kHz, at least 400 kHz, at least 500 kHz, at least 600 kHz, at least 700 kHz, at least 800 kHz, at least 900 kHz, at least 1 megahertz (MHz), at least 2 MHz, at least 3 MHz, at least 4 MHz, at least 5 MHz, at least 6 MHz, at least 7 MHz, at least 8 MHz, at least 9 MHz, at least 10 MHz, at least 20 MHz, at least 30 MHz, at least 40 MHz, at least 50 MHz, at least 60 MHz, at least 70 MHz, at least 80 MHz, at least 90 MHz, at least 100 MHz, at least 200 MHz, at least 300 MHz, at least 400 MHz, at least 500 MHz, at least 600 MHz, at least 700 MHz, at least 800 MHz, at least 900 MHz, at least 1,000 MHz, or more. The laser light may have a repetition rate of at most 1,000 MHz, at most 900 MHz, at most 800 MHz, at most 700 MHz, at most 600 MHz, at most 500 MHz, at most 400 MHz, at most 300 MHz, at most 200 MHz, at most 100 MHz, at most 90 MHz, at most 80 MHz, at most 70 MHz, at most 60 MHz, at most 50 MHz, at most 40 MHz, at most 30 MHz, at most 20 MHz, at most 10 MHz, at most 9 MHz, at most 8 MHz, at most 7 MHz, at most 6 MHz, at most 5 MHz, at most 4 MHz, at most 3 MHz, at most 2 MHz, at most 1 MHz, at most 900 kHz, at most 800 kHz, at most 700 kHz, at most 600 kHz, at most 500 kHz, at most 400 kHz, at most 300 kHz, at most 200 kHz, at most 100 kHz, at most 90 kHz, at most 80 kHz, at most 70 kHz, at most 60 kHz, at most 50 kHz, at most 40 kHz, at most 30 kHz, at most 20 kHz, at most 10 kHz, at most 9 kHz, at most 8 kHz, at most 7 kHz, at most 6 kHz, at most 5 kHz, at most 4 kHz, at most 3 kHz, at most 2 kHz, at most 1 kHz, at most 900 Hz, at most 800 Hz, at most 700 Hz, at most 600 Hz, at most 500 Hz, at most 400 Hz, at most 300 Hz, at most 200 Hz, at most 100 Hz, at most 90 Hz, at most 80 Hz, at most 70 Hz, at most 60 Hz, at most 50 Hz, at most 40 Hz, at most 30 Hz, at most 20 Hz, at most 10 Hz, at most 9 Hz, at most 8 Hz, at most 7 Hz, at most 6 Hz, at most 5 Hz, at most 4 Hz, at most 3 Hz, at most 2 Hz, at most 1 Hz, or less. The laser light may have a repetition rate that is within a range defined by any two of the preceding values.

The laser light may have a pulse energy of at least 1 nanojoule (nJ), at least 2 nJ, at least 3 nJ, at least 4 nJ, at least 5 nJ, at least 6 nJ, at least 7 nJ, at least 8 nJ, at least 9 nJ, at least 10 nJ, at least 20 nJ, at least 30 nJ, at least 40 nJ, at least 50 nJ, at least 60 nJ, at least 70 nJ, at least 80 nJ, at least 90 nJ, at least 100 nJ, at least 200 nJ, at least 300 nJ, at least 400 nJ, at least 500 nJ, at least 600 nJ, at least 700 nJ, at least 800 nJ, at least 900 nJ, at least 1 microjoule (μJ), at least 2 μJ, at least 3 μJ, at least 4 μJ, at least 5 μJ, at least 6 μJ, at least 7 μJ, at least 8 μJ, at least 9 μJ, at least 10 μJ, at least 20 μJ, at least 30 μJ, at least 40 μJ, at least 50 μJ, at least 60 μJ, at least 70 μJ, at least 80 μJ, at least 90 μJ, at least 100 μJ, at least 200 μJ, at least 300 μJ, at least 400 μJ, at least 500 μJ, at least 600 μJ, at least 700 μJ, at least 800 μJ, at least 900 μJ, a least 1 millijoule (mJ), at least 2 mJ, at least 3 mJ, at least 4 mJ, at least 5 mJ, at least 6 mJ, at least 7 mJ, at least 8 mJ, at least 9 mJ, at least 10 mJ, at least 20 mJ, at least 30 mJ, at least 40 mJ, at least 50 mJ, at least 60 mJ, at least 70 mJ, at least 80 mJ, at least 90 mJ, at least 100 mJ, at least 200 mJ, at least 300 mJ, at least 400 mJ, at least 500 mJ, at least 600 mJ, at least 700 mJ, at least 800 mJ, at least 900 mJ, a least 1 Joule (J), or more. The laser light may have a pulse energy of at most 1 J, at most 900 mJ, at most 800 mJ, at most 700 mJ, at most 600 mJ, at most 500 mJ, at most 400 mJ, at most 300 mJ, at most 200 mJ, at most 100 mJ, at most 90 mJ, at most 80 mJ, at most 70 mJ, at most 60 mJ, at most 50 mJ, at most 40 mJ, at most 30 mJ, at most 20 mJ, at most 10 mJ, at most 9 mJ, at most 8 mJ, at most 7 mJ, at most 6 mJ, at most 5 mJ, at most 4 mJ, at most 3 mJ, at most 2 mJ, at most 1 mJ, at most 900 μJ, at most 800 μJ, at most 700 μJ, at most 600 μJ, at most 500 μJ, at most 400 μJ, at most 300 μJ, at most 200 μJ, at most 100 μJ, at most 90 μJ, at most 80 μJ, at most 70 μJ, at most 60 μJ, at most 50 μJ, at most 40 μJ, at most 30 μJ, at most 20 μJ, at most 10 μJ, at most 9 μJ, at most 8 μJ, at most 7 μJ, at most 6 μJ, at most 5 μJ, at most 4 μJ, at most 3 μJ, at most 2 μJ, at most 1 μJ, at most 900 nJ, at most 800 nJ, at most 700 nJ, at most 600 nJ, at most 500 nJ, at most 400 nJ, at most 300 nJ, at most 200 nJ, at most 100 nJ, at most 90 nJ, at most 80 nJ, at most 70 nJ, at most 60 nJ, at most 50 nJ, at most 40 nJ, at most 30 nJ, at most 20 nJ, at most 10 nJ, at most 9 nJ, at most 8 nJ, at most 7 nJ, at most 6 nJ, at most 5 nJ, at most 4 nJ, at most 3 nJ, at most 2 nJ, at most 1 nJ, or less. The laser light may have a pulse energy that is within a range defined by any two of the preceding values. For instance, the laser light may have a pulse energy between 100 mJ and 500 mJ.

The laser light may have an average power of at least 1 microwatt (μV), at least 2 μW, at least 3 μW, at least 4 μW, at least 5 μW, at least 6 μW, at least 7 μW, at least 8 μW, at least 9 μW, at least 10 μW, at least 20 μW, at least 30 μW, at least 40 μW, at least 50 μW, at least 60 μW, at least 70 μW, at least 80 μW, at least 90 μW, at least 100 μW, at least 200 μW, at least 300 μW, at least 400 μW, at least 500 μW, at least 600 μW, at least 700 μW, at least 800 μW, at least 900 μW, at least 1 milliwatt (mW), at least 2 mW, at least 3 mW, at least 4 mW, at least 5 mW, at least 6 mW, at least 7 mW, at least 8 mW, at least 9 mW, at least 10 mW, at least 20 mW, at least 30 mW, at least 40 mW, at least 50 mW, at least 60 mW, at least 70 mW, at least 80 mW, at least 90 mW, at least 100 mW, at least 200 mW, at least 300 mW, at least 400 mW, at least 500 mW, at least 600 mW, at least 700 mW, at least 800 mW, at least 900 mW, at least 1 watt (W), at least 2 W, at least 3 W, at least 4 W, at least 5 W, at least 6 W, at least 7 W, at least 8 W, at least 9 W, at least 10 W, at least 20 W, at least 30 W, at least 40 W, at least 50 W, at least 60 W, at least 70 W, at least 80 W, at least 90 W, at least 100 W, at least 200 W, at least 300 W, at least 400 W, at least 500 W, at least 600 W, at least 700 W, at least 800 W, at least 900 W, at least 1,000 W, or more. The laser light may have an average power of at most 1,000 W, at most 900 W, at most 800 W, at most 700 W, at most 600 W, at most 500 W, at most 400 W, at most 300 W, at most 200 W, at most 100 W, at most 90 W, at most 80 W, at most 70 W, at most 60 W, at most 50 W, at most 40 W, at most 30 W, at most 20 W, at most 10 W, at most 9 W, at most 8 W, at most 7 W, at most 6 W, at most 5 W, at most 4 W, at most 3 W, at most 2 W, at most 1 W, at most 900 mW, at most 800 mW, at most 700 mW, at most 600 mW, at most 500 mW, at most 400 mW, at most 300 mW, at most 200 mW, at most 100 mW, at most 90 mW, at most 80 mW, at most 70 mW, at most 60 mW, at most 50 mW, at most 40 mW, at most 30 mW, at most 20 mW, at most 10 mW, at most 9 mW, at most 8 mW, at most 7 mW, at most 6 mW, at most 5 mW, at most 4 mW, at most 3 mW, at most 2 mW, at most 1 mW, at most 900 μW, at most 800 μW, at most 700 μW, at most 600 μW, at most 500 μW, at most 400 μW, at most 300 μW, at most 200 μW, at most 100 μW, at most 90 μW, at most 80 μW, at most 70 μW, at most 60 μW, at most 50 μW, at most 40 μW, at most 30 μW, at most 20 μW, at most 10 μW, at most 9 μW, at most 8 μW, at most 7 μW, at most 6 μW, at most 5 μW, at most 4 μW, at most 3 μW, at most 2 μW, at most 1 μW, or more. The laser light may have a power that is within a range defined by any two of the preceding values.

The laser light may comprise a wavelength in the ultraviolet (UV), visible, or infrared (IR) portions of the electromagnetic spectrum. The laser light may comprise a wavelength of at least 100 nanometers (nm), at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 260 nm, at least 270 nm, at least 280 nm, at least 290 nm, at least 300 nm, at least 310 nm, at least 320 nm, at least 330 nm, at least 340 nm, at least 350 nm, at least 360 nm, at least 370 nm, at least 380 nm, at least 390 nm, at least 400 nm, at least 410 nm, at least 420 nm, at least 430 nm, at least 440 nm, at least 450 nm, at least 460 nm, at least 470 nm, at least 480 nm, at least 490 nm, at least 500 nm, at least 510 nm, at least 520 nm, at least 530 nm, at least 540 nm, at least 550 nm, at least 560 nm, at least 570 nm, at least 580 nm, at least 590 nm, at least 600 nm, at least 610 nm, at least 620 nm, at least 630 nm, at least 640 nm, at least 650 nm, at least 660 nm, at least 670 nm, at least 680 nm, at least 690 nm, at least 700 nm, at least 710 nm, at least 720 nm, at least 730 nm, at least 740 nm, at least 750 nm, at least 760 nm, at least 770 nm, at least 780 nm, at least 790 nm, at least 800 nm, at least 810 nm, at least 820 nm, at least 830 nm, at least 840 nm, at least 850 nm, at least 860 nm, at least 870 nm, at least 880 nm, at least 890 nm, at least 900 nm, at least 910 nm, at least 920 nm, at least 930 nm, at least 940 nm, at least 950 nm, at least 960 nm, at least 970 nm, at least 980 nm, at least 990 nm, at least 1,000 nm, at least 1,010 nm, at least 1,020 nm, at least 1,030 nm, at least 1,040 nm, at least 1,050 nm, at least 1,060 nm, at least 1,070 nm, at least 1,080 nm, at least 1,090 nm, at least 1,100 nm, at least 1,110 nm, at least 1,120 nm, at least 1,130 nm, at least 1,140 nm, at least 1,150 nm, at least 1,160 nm, at least 1,170 nm, at least 1,180 nm, at least 1,190 nm, at least 1,200 nm, at least 1,210 nm, at least 1,220 nm, at least 1,230 nm, at least 1,240 nm, at least 1,250 nm, at least 1,260 nm, at least 1,270 nm, at least 1,280 nm, at least 1,290 nm, at least 1,300 nm, at least 1,310 nm, at least 1,320 nm, at least 1,330 nm, at least 1,340 nm, at least 1,350 nm, at least 1,360 nm, at least 1,370 nm, at least 1,380 nm, at least 1,390 nm, at least 1,400 nm, or more. The laser light may comprise a wavelength of at most 1,400 nm, at most 1,390 nm, at most 1,380 nm, at most 1,370 n, at most 1,360 nm, at most 1,350 nm, at most 1,340 nm, at most 1,330 nm, at most 1,320 nm, at most 1,310 nm, at most 1,300 nm, at most 1,290 nm, at most 1,280 nm, at most 1,270 n, at most 1,260 nm, at most 1,250 nm, at most 1,240 nm, at most 1,230 nm, at most 1,220 nm, at most 1,210 nm, at most 1,200 nm, at most 1,190 nm, at most 1,180 nm, at most 1,170 n, at most 1,160 nm, at most 1,150 nm, at most 1,140 nm, at most 1,130 nm, at most 1,120 nm, at most 1,110 nm, at most 1,100 nm, at most 1,090 nm, at most 1,080 nm, at most 1,070 n, at most 1,060 nm, at most 1,050 nm, at most 1,040 nm, at most 1,030 nm, at most 1,020 nm, at most 1,010 nm, at most 1,000 nm, at most 990 nm, at most 980 nm, at most 970 nm, at most 960 nm, at most 950 nm, at most 940 nm, at most 930 nm, at most 920 nm, at most 910 nm, at most 900 nm, at most 890 nm, at most 880 nm, at most 870 nm, at most 860 nm, at most 850 nm, at most 840 nm, at most 830 nm, at most 820 nm, at most 810 nm, at most 800 nm, at most 790 nm, at most 780 nm, at most 770 nm, at most 760 nm, at most 750 nm, at most 740 nm, at most 730 nm, at most 720 nm, at most 710 nm, at most 700 nm, at most 690 nm, at most 680 nm, at most 670 nm, at most 660 nm, at most 650 nm, at most 640 nm, at most 630 nm, at most 620 nm, at most 610 nm, at most 600 nm, at most 590 nm, at most 580 nm, at most 570 nm, at most 560 nm, at most 550 nm, at most 540 nm, at most 530 nm, at most 520 nm, at most 510 nm, at most 500 nm, at most 490 nm, at most 480 nm, at most 470 nm, at most 460 nm, at most 450 nm, at most 440 nm, at most 430 nm, at most 420 nm, at most 410 nm, at most 400 nm, at most 390 nm, at most 380 nm, at most 370 nm, at most 360 nm, at most 350 nm, at most 340 nm, at most 330 nm, at most 320 nm, at most 310 nm, at most 300 nm, at most 290 nm, at most 280 nm, at most 270 nm, at most 260 nm, at most 250 nm, at most 240 nm, at most 230 nm, at most 220 nm, at most 210 nm, at most 200 nm, at most 190 nm, at most 180 nm, at most 170 nm, at most 160 nm, at most 150 nm, at most 140 nm, at most 130 nm, at most 120 nm, at most 110 nm, at most 100 nm, or less. The laser light may comprise a wavelength that is within a range defined by any two of the preceding values.

The laser light may have a bandwidth of at least 0.001 nm, at least 0.002 nm, at least 0.003 nm, at least 0.004 nm, at least 0.005 nm, at least 0.006 nm, at least 0.007 nm, at least 0.008 nm, at least 0.009 nm, at least 0.01 nm, at least 0.02 nm, at least 0.03 nm, at least 0.04 nm, at least 0.05 nm, at least 0.06 nm, at least 0.07 nm, at least 0.08 nm, at least 0.09 nm, at least 0.1 nm, at least 0.2 nm, at least 0.3 nm, at least 0.4 nm, at least 0.5 nm, at least 0.6 nm, at least 0.7 nm, at least 0.8 nm, at least 0.9 nm, at least 1 nm, at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, or more. The laser light may have a bandwidth of at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, at most 0.9 nm, at most 0.8 nm, at most 0.7 nm, at most 0.6 nm, at most 0.5 nm, at most 0.4 nm, at most 0.3 nm, at most 0.2 nm, at most 0.1 nm, at most 0.09 nm, at most 0.08 nm, at most 0.07 nm, at most 0.06 nm, at most 0.05 nm, at most 0.04 nm, at most 0.03 nm, at most 0.02 nm, at most 0.01 nm, at most 0.009 nm, at most 0.008 nm, at most 0.007 nm, at most 0.006 nm, at most 0.005 nm, at most 0.004 nm, at most 0.003 nm, at most 0.002 nm, at most 0.001 nm, or less. The laser light may have a bandwidth that is within a range defined by any two of the preceding values.

The laser light may have a diameter (for instance, as measured by a Rayleigh beam width, full width at half maximum, l/e² width, second moment width, knife-edge width, D86 width, or any other measure of beam diameter) of at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, at least 6 mm, at least 7 mm, at least 8 mm, at least 9 mm, at least 10 mm, at least 20 mm, at least 30 mm, at least 40 mm, at least 50 mm, at least 60 mm, at least 70 mm, at least 80 mm, at least 90 mm, at least 100 mm, or more. The first light may have a diameter of at most 100 mm, at most 90 mm, at most 80 mm, at most 70 mm, at most 60 mm, at most 50 mm, at most 40 mm, at most 30 mm, at most 20 mm, at most 10 mm, at most 9 mm, at most 8 mm, at most 7 mm, at most 6 mm, at most 5 mm, at most 4 mm, at most 3 mm, at most 2 mm, at most 1 mm, at most 0.9 mm, at most 0.8 mm, at most 0.7 mm, at most 0.6 mm, at most 0.5 mm, at most 0.4 mm, at most 0.3 mm, at most 0.2 mm, at most 0.1 mm, or less. The laser light may have a diameter that is within a range defined by any two of the preceding values. In some cases, the laser light may have a diameter that is smaller than the diameter of a wearable ocular device. In some instances, the laser light may have a diameter that is approximately equal to the diameter of a wearable ocular device. In still further instances, the laser light may have a diameter that is larger than the diameter of a wearable ocular device. For instance, the laser light may have a diameter that allows the laser light to simultaneously illuminate a plurality of wearable ocular devices. Such a system may allow the simultaneous production of diffraction gratings on a plurality of wearable ocular devices in a batch process.

XI. Computer Systems

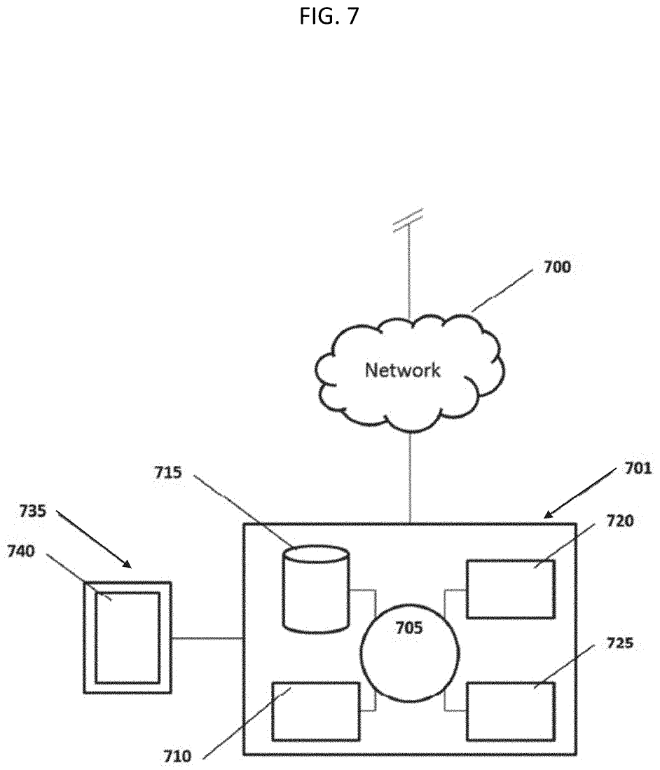

The present disclosure provides computer systems for implementing methods and devices of the present disclosure. FIG. 7 shows a computer system 701 that is programmed or otherwise configured to operate any method or system described herein (such as any method of imparting color to a wearable ocular device described herein). The computer system 701 can regulate various aspects of the present disclosure. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine-readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, enact any of the methods for imparting color to a wearable ocular device as described herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1: Cosmetic Enhancements for Use in Movies

Systems and methods of the present disclosure may be utilized to provide cosmetic enhancements to the eyes of actors in movies. For instance, a contact lens may be manufactured using the systems and methods described herein to create the appearance that a wearer of the contact lens has the eyes of an animal or monster. The contact lens may be worn by an actor during filming of a movie in order to provide a more realistic depiction of the animal or monster.

### Example 2: System Using Multiple Aperture Wheels

Systems and methods of the present disclosure may utilize multiple aperture wheels to provide various aperture patterns for pattern interference ablation on a surface of a substrate. In an exemplary embodiment, as depicted by FIG. 3, each aperture wheel comprises multiple aperture patterns 332 and at least one window 333. In an example embodiment, each aperture wheel may comprise six aperture patterns and one window. If five aperture wheels are provided, when the system may utilize 30 different aperture patterns provided on the plurality of aperture wheels. Tracking of the aperture wheels by the encoder system 360 may allow for proper adjustment of the aperture wheels.

In an example embodiment, if the selected aperture pattern is provided on a second aperture wheel 336, then the rotation of the other aperture wheels (334, 337, 338, and 339) may be adjusted such that the windows of the other aperture wheels are aligned with the selected aperture pattern of the second wheel 336 when the aperture pattern is aligned with the optical path of the laser light. Therefore, the window 333 of first aperture wheel 334 may not modify the laser light prior to passing through the selected aperture of the second wheel 336. Further, the windows of the succeeding aperture wheels (337, 338, and 339) will not further modify the interference pattern created by the selected aperture provided on the second wheel 336.

In some embodiments, wherein multiple interference patterns are to be ablated on a substrate, the aperture wheels may be adjusted such that a second aperture pattern is selected to create an interference pattern to be ablated onto a surface of a device. The device may be provided on a movable stage and may be moved between ablations of multiple interference patterns. In an example, a second selected aperture pattern may be provided on the third aperture wheel 337. Then, the rotation of the other aperture wheels (334, 336, 338, and 339) may be adjusted such that the windows of the other aperture wheels are aligned with the second selected aperture pattern of the third wheel 336 when the aperture pattern is aligned with the optical path of the laser light. Therefore, the window 333 of first aperture wheel 334 and window of the second aperture wheel 336 may not modify the laser light prior to passing through the second selected aperture of the third wheel 337. Further, the windows of the succeeding aperture wheels (338, and 339) will not further modify the interference pattern created by the second selected aperture provided on the third wheel 337.

The process may be repeated using any of the aperture patterns on any of the provided aperture wheels to impart a desired representation or pattern onto a surface of a device.

### Example 3: Patterning of a Dehydrated Surface

In some embodiments, interference patterns created by the aperture patterns described herein are ablated onto a dehydrated device. In some embodiments the dehydrated device is a dehydrated contact lens. In some embodiments, the interference patterns are ablated onto the surface of the dehydrated lens to form a diffraction grating.

In some embodiments, when the lenses are hydrated, the grating spacing will increase by a known amount, depending on the water content of the contact lens. In some embodiments, the water content in contact lenses varies from 38% to 75% of the overall weight of the contact lens. In some embodiments, the water content is less than 40% for low water content lenses, 50%-60% for medium water content lenses and over 60% for high water content lenses. In some embodiments, the ablation process on a contact lens is carried out on a dehydrated silicone hydrogel contact. When water is added, the lens may expand according to the following equation:

% of Linear Expansion=−0.9+0.5θ_{X}(% H₂O)

In an exemplary embodiment, a grating that diffracts green may be imparted on a lens with a 50% isotropic linear expansion. In an embodiment, a green (λ=550 nm) reconstruction light with a θ=25 degree illumination. This means that the final grating spacing (d) from the grating equation: 2d sin θ=λ, may be 650 nm. Since the dehydrated lens may expand by 50%, the grating on the dehydrated lens should half of that, or d=325 nm. A λ=532 nm short pulse laser may be used for ablating the grating, and the angle (θ) between the laser beams to make the grating that expands to the correct size for the green reconstruction light is equal to 54.9 degrees. The sin of half this angle may provide the NA (numerical aperture) of the optics to make this grating from two apertures in front of it. In some embodiments, that would be an NA=0.46. In some embodiments, that corresponds to approximately a 20× microscope objective to make the grating.

What is claimed is:

1. A system for imparting a pattern onto a surface, the system comprising:
a laser for emitting a laser beam along an optical path to the surface; and
an aperture substrate comprising one or more aperture patterns to be placed in the optical path;
wherein emission of the laser beam is coordinated with a position of the aperture substrate such that the laser beam is modified by the one or more aperture patterns to impart at least a portion of the pattern onto the surface.

2. The system of claim 1, further comprising an encoder configured to track the position of the aperture substrate; and a controller for coordinating the emission of the laser beam with the position of the aperture substrate.

3. The system of claim 2, wherein the aperture substrate rotates at a rate of about 3,000 to 6,000 rotations per minute.

4. The system of claim 2, further comprising a moveable stage, wherein the surface is provided on the moveable stage, and wherein the controller coordinates movement of the moveable stage with the emission of the laser beam.

5. The system of claim 1, wherein the surface is a surface of a dehydrated hydrogel contact lens, and wherein the one or more aperture patterns are configured to account for an expansion of the dehydrated hydrogel contact lens during hydration.

6. The system of claim 1, further comprising a focal lens comprised of one or more optical elements to focus the modified laser beam onto the surface, wherein the focal lens is placed into the optical path after the laser beam is modified by the one or more aperture patterns.

7. The system of claim 1, further comprising a beam expander, wherein the beam expander is placed into the optical path prior to the aperture substrate.

8. A system for imparting a pattern onto a surface, the system comprising:
a laser for emitting a laser beam along an optical path to the surface;
a plurality of rotatable aperture wheels, each aperture wheel comprising one or more aperture patterns; and
an encoder system configured to track the position of the plurality of rotatable aperture wheels,
wherein emission of the laser beam is synchronized with rotation of the plurality of aperture wheels such that the laser beam is modified by the one or more aperture patterns to impart at least a portion of the pattern onto the surface.

9. The system of claim 8, wherein the surface is a surface of a wearable ocular device.

10. The system of claim 8, wherein the surface is a surface of a dehydrated hydrogel contact lens, and wherein the one or more aperture patterns are configured to account for an expansion of the dehydrated hydrogel contact lens during hydration.

11. The system of any claim 8, wherein each rotatable aperture wheel comprises a window such that light passing through the window is not modified.

12. The system of claim 8, further comprising a focal lens comprised of one or more optical elements to focus the modified laser beam onto the surface, wherein the focal lens is placed into the optical path after the laser beam is modified by the one or more aperture patterns.

13. The system of claim 8, further comprising a beam expander comprised of one or more optical elements, wherein the beam expander is placed into the optical path prior to the plurality of aperture wheels.

14. The system of claim 8, wherein each rotatable aperture wheel rotates at a rate of about 3,000 to 6,000 rotations per minute.

15. The system of claim 14, wherein the rate of rotation of each aperture wheel is individually varied.

16. A method for imparting a pattern on to a surface, comprising:
a) positioning an aperture substrate along an optical path of a laser beam, the aperture substrate comprising one or more aperture patterns;
b) selecting an aperture pattern of the one or more aperture patterns to modify the laser beam;
c) rotating the aperture substrate; and
d) emitting the laser beam along the optical path to the surface when the selected aperture pattern is aligned with the optical path,
wherein the one or more aperture patterns are configured to modify the laser beam into a light pattern and impart at least a portion of the pattern onto the surface.

17. The method of claim 16 further comprising a step of applying an optically absorptive material to the surface prior to the step of emitting the laser beam.

18. The method of claim 16, further comprising repeating steps (a)-(d) to impart the pattern onto the surface.

19. The method of claim 16, wherein the surface is a surface of a wearable ocular device.

20. The method of claim 19, wherein the wearable ocular device is a contact lens, and wherein the contact lens is a dehydrated hydrogel contact lens.
